(19) United States
(12) Patent Application Publication
(10) Pub. No.: US 2026/0183392 A1
(43) Pub. Date: Jul. 2, 2026

(54) METHODS AND KITS FOR DNA EDITING AND TREATING, AMELIORATING AND/OR PREVENTING CANCER

(71) Applicant: FC IP Holdings, LLC, Dover, DE (US)

(72) Inventors: Hao Wu, Lexington, MA (US); Richard Shan, Cambridge, MA (US); Keqiang Xie, Cambridge, MA (US)

(73) Assignee: FC IP HOLDINGS, LLC, Dover, DE (US)

(21) Appl. No.: 19/133,020

(22) PCT Filed: Nov. 28, 2023
(86) PCT No.: PCT/US23/81394

§ 371 Date: May 27, 2025

Related U.S. Application Data
(60) Provisional application No. 63/428,996, filed on Nov. 30, 2022.

(51) Int. Cl. A61K 40/11; A61K 40/31; A61K 40/42; A61P 35/00; C12N 9/22; C12N 15/11; C12N 15/90

(52) U.S. Cl. CPC A61K 40/11; A61K 40/31; A61K 40/4211; A61K 40/4212; A61P 35/00; C12N 9/226; C12N 15/11; C12N 15/907; C12N 2310/20

(57) ABSTRACT

Described herein is a method of editing a DNA in cells. The method includes delivering into the cells a circular single-stranded DNA (cssDNA) donor template, and a DNA editing construct and/or a nucleic acid encoding the DNA editing construct. The DNA editing constructs inserts a DNA insert in the cssDNA donor template into the DNA in the cells via homologous recombination and/or homology directed repair. The length of the DNA insert is about 2,000 bases or longer, and the efficiency of the editing is 20% or higher based on the total number of the cells. Also described are kits for performing the DNA editing method, as well as methods of killing cancer cells and/or treating cancer with CAR-T cells engineered using the DNA editing method.

### CROSS-REFERENCE TO RELATED APPLICATIONS

The present application claims priority under 35 U.S.C. § 119 (e) to U.S. Provisional Patent Application No. 63/428,996, filed Nov. 30, 2022, which is incorporated herein by reference in its entirety.

### REFERENCE TO AN ELECTRONIC SEQUENCE LISTING

The present application contains a Sequence Listing, which has been submitted in XML (ST.26) format and is incorporated herein by reference in its entirety. Said XML copy, created on Nov. 21, 2023, is named 385722-1002WO1_Seq_Listing.xml and is 10,822 bytes in size.

### BACKGROUND

Unsatisfactory editing efficiency has always been an issue in the art of gene editing. Therefore, there is a need for DNA editing constructs and/or methods that can improve editing efficiency. The present invention addresses this need.

Chimeric antigen receptor T-cells (CAR-T cells) have been used to treat cancers. Currently, CAR-T cells are generally engineered from primary T-cells using viral vectors such as those derived from lentivirus or adeno-associated virus type 6 (AAV6). Viral vectors, however, have reported safety issues, manufacturing constraints, and restricted applications due to its packaging limit. Thus, there is a need for novel cancer treatments involving improved CAR-T cell engineering that overcome the shortcomings of the viral vectors. The present invention addresses this need, as well.

### SUMMARY

In some aspects, the present invention is directed to the following non-limiting embodiments:

### Method of Editing a DNA

In some aspects, the present invention is directed to a method of editing a DNA in cells.

In some embodiments, the method comprises delivering into the cells: a circular single-stranded DNA (cssDNA) donor template; and a DNA editing construct, or a nucleic acid encoding the DNA editing construct.

In some embodiments, the cssDNA comprises: a DNA insert; a 5′-homology arm; and a 3′-homology arm. In some embodiments, the 5′ homology arm and the 3′ homology arm are complementary to the DNA in a target region.

In some embodiments, the DNA editing construct has a site-specific DNA endonuclease activity.

In some embodiments, the DNA editing construct introduces a DNA break in the target region of the DNA.

In some embodiments, the DNA insert is inserted into the target region via homologous recombination (HR), or homology directed repair (HDR).

In some embodiments, the length of the DNA insert is about 2,000 bases or longer and editing efficiency is 20% or higher based on a total number of the cells, or the length of the DNA insert is less than about 2,000 bases and editing efficiency is 30% or higher.

In some embodiments, the cells are at least one selected from the group consisting of induced pluripotent stem cells (iPSCs), hematopoietic stem cells (HSCs), B-cells, and T-cells.

In some embodiments, the cells are iPSCs, the length of the DNA insert is about 2,000 bases or longer, and the editing efficiency is 40% or higher based on a total number of the cells.

In some embodiments, the cells are hematopoietic stem cells (HSCs), the length of the DNA insert is about 2,000 bases or longer, and the editing efficiency is 40% or higher based on a total number of the cells.

In some embodiments, the cells are B-cells, the length of the DNA insert is about 2,000 bases or longer, and the editing efficiency is 20% or higher based on a total number of the cells.

In some embodiments, the cells are T-cells, the length of the DNA insert is about 2,000 bases or longer, and the editing efficiency is 40% or higher based on the total number of cells.

In some embodiments, each of the 5′-homology arm and the 3′-homology arm independently has a length ranging from about 25 nucleotides to about 5000 nucleotides.

In some embodiments, the DNA editing construct is or comprises at least one site-specific nuclease selected from the group consisting of a Cas nuclease, a zinc-finger nuclease, a meganuclease, and a transcription activator-like effector nuclease (TALEN).

In some embodiments, the DNA editing construct is or comprises a class I Cas protein or a class II Cas protein.

In some embodiments, the DNA editing construct is or comprises Cas9, Cas3, Cas10, Cas11, CasX, Cas12a, MAD7, or catalytic dead forms or nickase forms thereof.

In some embodiments, the DNA is a genomic DNA.

In some embodiments, the cells comprise two or more copies of the DNA, and 20% or more of the edited cells are edited in two or more copies of the DNA.

In some embodiments, the construct and/or nucleic acid and the ssDNA are delivered into the cell by a viral vector, a lipid or non-lipid nanoparticle delivery, an exosome delivery, an electroporation delivery, a gene gun delivery, or an injection.

In some embodiments, the method further comprises contacting the cells with a nonhomologous end joining (NHEJ) inhibitor or a DNA recombinase stimulator.

In some embodiments, the NHEJ inhibitor is a DNA-dependent protein kinase (DNA-PK) inhibitor.

In some embodiments, the DNA-PK comprises M-3814, Alt-R HDR enhancer, Alt-R HDR enhancer V2, generic DNA ligase inhibitor comprises SCR7, generic DNA recombinase stimulator comprises RS-1, or combinations thereof.

### Kit

In some aspects, the present invention is directed to a kit for editing a DNA.

In some embodiments, the kit comprises: a circular single-stranded DNA (cssDNA) donor template comprising a DNA insert; a 5′-homology arm; and a 3′-homology arm, wherein the 5′ homology arm and the 3′ homology arm are complementary to the DNA in a target region; a DNA editing construct or a nucleic acid encoding the DNA editing construct, wherein the DNA editing construct has a site-specific DNA endonuclease activity, and wherein the DNA editing construct introduces a DNA break in the target region of the DNA; and a nonhomologous end joining (NHEJ) inhibitor or a DNA recombinase stimulator.

In some embodiments, each of the 5′-homology arm and the 3′-homology arm of the cssDNA donor template independently has a length ranging from about 25 nucleotides to about 5000 nucleotides.

In some embodiments, the DNA editing construct is or comprises at least one site-specific nuclease selected from the group consisting of a Cas nuclease, a zinc-finger nuclease, a meganuclease, and a transcription activator-like effector nuclease (TALEN).

In some embodiments, the DNA editing construct is or comprises a class I Cas protein or a class II Cas protein.

In some embodiments, the DNA editing construct is or comprises Cas9, Cas3, Cas10, Cas11, CasX, Cas12a, MAD7, or catalytic dead forms or nickase forms thereof.

In some embodiments, the DNA is a genomic DNA.

In some embodiments, the NHEJ inhibitor is a DNA-dependent protein kinase (DNA-PK) inhibitor.

In some embodiments, the DNA-PK comprises M-3814, Alt-R HDR enhancer, Alt-R HDR enhancer V2, generic DNA ligase inhibitor comprises SCR7, generic DNA recombinase stimulator comprises RS-1.

In some embodiments, the kit further comprises a component for delivering the DNA editing construct and/or the nucleic acid encoding the DNA editing construct, and the ssDNA, wherein the component comprises a microinjector, a lipid for forming lipid nanoparticles, a exosome, a gene gun, a metal nanoparticle, or a cuvette for electroporation.

### Method of Killing Cancer Cell

In some aspects, the present invention is directed to a method of killing cancer cell.

In some embodiments, the method comprising contacting the cancer cell with an engineered T-cell comprising an engineered T-cell receptor (TCR) and/or a chimeric antigen receptor (CAR) targeting the cancer cell.

In some embodiments, the engineered T-cell is prepared by delivering into a T-cell: a circular single-stranded DNA (cssDNA) donor template; and a DNA editing construct or a nucleic acid encoding the DNA editing construct.

In some embodiments, the cssDNA comprises: a DNA insert; a 5′-homology arm; and a 3′-homology arm.

In some embodiments, the 5′ homology arm and the 3′ homology arm are complementary to the DNA in a target region.

In some embodiments, the DNA editing construct has a site-specific DNA endonuclease activity, and the DNA editing construct introduces a DNA break in the target region of the DNA.

In some embodiments, the DNA insert is inserted into the target region via homologous recombination (HR), or homology directed repair (HDR).

In some embodiments, the method further comprises preparing the engineered T-cell, and preparing the engineered T-cell comprises delivering into the T-cell the cssDNA donor template, and the DNA editing construct and/or the nucleic acid encoding the DNA editing construct.

In some embodiments, the insertion of the DNA insert produces a chimeric antigen receptor targeting at least one selected from the group consisting of B-cell maturation antigen (BCMA), carcinoembryonic antigen (CEA), CD2, CD4, CD5, CD7, CD19, CD22, CD30, claudin 18.2 (CLDN 18.2), epithelial cellular adhesion molecule (EpCAM), folate receptor 1 (FOLR1), human epidermal growth factor receptor 2 (HER2), mesothelin (MSLN), mucin 1 (MUC1), natural killer group 2D (NKG2D).

In some embodiments, the target region of the DNA is a B2M locus, a genomic safe harbor locus (such as an AAVS1 locus a CCR5 locus, a hRosa26 locus, a Rogi1 locus, a Rogi2 locus, a GSH1-6 locus, a PD1 locus, a TRAC locus, or combinations thereof.

In some embodiments, each of the 5′-homology arm and the 3′-homology arm independently has a length ranging from about 25 nucleotides to about 5000 nucleotides.

In some embodiments, the DNA editing construct is or comprises at least one site-specific nuclease selected from the group consisting of a Cas nuclease, a zinc-finger nuclease, a meganuclease, and a transcription activator-like effector nuclease (TALEN).

In some embodiments, the DNA editing construct is or comprises a class I Cas protein or a class II Cas protein.

In some embodiments, the DNA editing construct is or comprises Cas9, Cas3, Cas10, Cas11, CasX, Cas12a, MAD7, or catalytic dead forms or nickase forms thereof.

In some embodiments, the cancer cell is a leukemia cancer cell, such as an acute lymphoblastic leukemia cancer cell.

In some embodiments, the cancer cell is a cancer cell line, a primary cancer cell, or a cancer cell in the body of a subject.

In some embodiments, the cancer cell is in the body of the subject, and wherein the subject is a mammal, such as a human.

Method of Treating, Ameliorating and/or Preventing Cancer

In some aspects, the present invention is directed to a method of treating, ameliorating and/or preventing cancer in a subject in need thereof, the method comprising administering to the subject an effective amount of engineered T-cells comprising an engineered T-cell receptor or a chimeric antigen receptor targeting the cancer.

In some embodiments, the CAR-T cell is prepared by delivering into a T-cell: a circular single-stranded DNA (cssDNA) donor template; and a DNA editing construct or a nucleic acid encoding the DNA editing construct.

In some embodiments, the cssDNA comprises: a DNA insert; a 5′-homology arm; and a 3′-homology arm.

In some embodiments, the 5′ homology arm and the 3′ homology arm are complementary to the DNA in a target region.

In some embodiments, the DNA editing construct has a site-specific DNA endonuclease activity, and wherein the DNA editing construct introduces a DNA break in the target region of the DNA.

In some embodiments, the DNA insert is inserted into the target region via homologous recombination (HR), or homology directed repair (HDR).

In some embodiments, the method further comprises preparing the engineered T-cells, and preparing the engineered T-cells comprises delivering into the T-cell the cssDNA donor template, and the DNA editing construct and/or the nucleic acid encoding the DNA editing construct.

In some embodiments, the insertion of the DNA insert produces a chimeric antigen receptor targeting at least one selected from the group consisting of B-cell maturation antigen (BCMA), carcinoembryonic antigen (CEA), CD2, CD4, CD5, CD7, CD19, CD22, CD30, claudin 18.2 (CLDN 18.2), epithelial cellular adhesion molecule (EpCAM), folate receptor 1 (FOLR1), human epidermal growth factor receptor 2 (HER2), mesothelin (MSLN), mucin 1 (MUC1), natural killer group 2D (NKG2D).

In some embodiments, the target region of the DNA is a B2M locus, a genomic safe harbor locus (such as an AAVS1 locus, hRosa26, Rogi1, Rogi2, GSH1-6 and/or a CCR5 locus), a PD1 locus, a TRAC locus, or combinations thereof.

In some embodiments, each of the 5′-homology arm and the 3′-homology arm independently has a length ranging from about 25 nucleotides to about 5000 nucleotides.

In some embodiments, the DNA editing construct is or comprises at least one site-specific nuclease selected from the group consisting of a Cas nuclease, a zinc-finger nuclease, a meganuclease, and a transcription activator-like effector nuclease (TALEN).

In some embodiments, the DNA editing construct is or comprises a class I Cas protein or a class II Cas protein.

In some embodiments, the DNA editing construct is or comprises Cas9, Cas3, Cas10, Cas11, CasX, Cas12a, MAD7, or catalytic dead forms or nickase forms thereof.

In some embodiments, the cancer is a leukemia cancer, such as an acute lymphoblastic leukemia cancer or B-cell non-Hodgkin lymphoma.

In some embodiments, the subject is a mammal, such as a human.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIGS. 1A-1E: Scalable cssDNA manufacturing and purification from bacteriophage, in accordance with some embodiments. FIG. 1A: Schematic diagram of cssDNA manufacturing. Recombinant phagemid containing sequence of interest was co-transformed with M13 helper plasmid into E. coli. Phage particles were produced and collected from culture supernatant. cssDNA was then extracted and purified from phage. FIG. 1B: Representative DNA agarose gel images showing the purity of manufactured cssDNA at lengths ranging from 1 Kb to 9 Kb. FIG. 1C: cssDNA yields (mg per liter of culture volume) for ˜100 of cssDNA molecules at various lengths. FIG. 1D: The violin plot of the production of same cssDNA molecules over different batches. FIG. 1E: cssDNA and dsDNA half-life analysis in K562 cells. cssDNA or dsDNA were electroporated into K562 cells. The remaining DNAs in cells were analyzed by quantitative PCR. DNA was normalized with 2^{ΔCT} methods. Half-life (T_{1/2}) were obtained after one phase decay non-linear curve fitting.

FIGS. 2A-2F: K562 engineering according to some embodiments. FIG. 2A: Schematic diagram of the CRISPR-mediated genomic integration of a GFP reporter gene at RAB11A locus with dsDNA, cssDNA or lssDNA donor templates. FIG. 2B: The GFP reporter knock-in efficiency at RAB11A locus in K562 cells after engineering with cssDNA, lssDNA or dsDNA as donor templates on Day 7 post electroporation. FIG. 2C: Cell viability of K562 cells on Day 4 after engineering with cssDNA, lssDNA or dsDNA as donor templates. FIG. 2D: Knock-in efficiency of GFP reporter gene (2 Kb payload) at RAB11A locus in K562 cells over time with increasing amount of cssDNA donor templates. FIG. 2E: Knock-in efficiency and cell viability with increasing amount of cssDNA donor templates in K562 cells. 2 Kb GFP reporter payload cssDNA targeting RAB11A locus was used to engineer K562 cells. Knock-in efficiency was measured on Day 7 and cell viability was assessed on Day 4 after electroporation. FIG. 2F: Schematic diagram of the 4 Kb and 8 Kb GFP reporter payload cssDNAs targeting RAB11A locus (upper panel). Knock-in efficiency (measured on Day 7) for the 4 Kb payload (middle) and 8 Kb payload (lower panels) with increasing donor template concentrations.

FIGS. 3A-3H: induced pluripotent stem cells (iPSC) engineering according to some embodiments. FIG. 3A: GFP knock-in efficiency (on Day 7) at RAB11A locus in iPSC using cssDNA or dsDNA as donor templates. FIG. 3B: Cell viability of cssDNA- and dsDNA-engineered iPSC as measured on Day 3. FIG. 3C: Does-dependent GFP reporter knock-in using GFP reporter cssDNA targeting RAB11A or B2M locus. FIG. 3D: GFP fluorescence and phase contrast imaging of representative engineered iPSC colony. FIG. 3E: Schematic diagram of cssDNA donor templates for multiplexed reporter gene knock-in at RAB11A and B2M loci. FIG. 3F: Dual knock-in of mCherry and GFP report genes at the same RAB11A locus. FIG. 3G: Dual knock-in of mCherry and GFP report genes at RAB11A locus and B2M locus, respectively. FIG. 3H: NHEJ small molecule inhibitors increased genome knock-in efficiency in iPS cells (left) without impacting cell health (right). iPSCs were treated with NHEJ small molecule at specified concentration for 24 hours. GFP knock-in efficiency was assessed on Day 7 post electroporation by flow cytometry. Cell viability was measured on Day 3 post electroporation. ****, p<0.0001 One-Way ANOVA Bonferroni Post Hoc Test between indicated group.

FIGS. 4A-4D: Reporter knock-in in human primary B/HSC cells, in accordance with some embodiments. FIG. 4A: GFP reporter knock-in efficiency at RAB11A locus in human primary B cells using cssDNA or dsDNA as donor templates. The electroporation was performed in Lonza Amaxa 4D-Nucleofector™ system. The electroporation was performed using MaxCyte ATX system. FIG. 4B: The time- and dose-dependent GFP knock-in efficiency at RAB11A locus using cssDNA as donor template with two different HSC electroporation programs. FIG. 4B: Quantification of the GFP knock-in efficiency at RAB11A locus in human primary B cells. FIGS. 4C-4D: GFP reporter knock in efficiency at RAB11A locus in human primary CD34″ hematopoietic stem and progenitor cells (HSPC) using cssDNA or dsDNA as donor templates. The electroporation was performed using MaxCyte ATX system. GFP knock-in efficiency was examined on Day 7 post electroporation by flow cytometry. Cell viability was measured on Day 3 post electroporation.

FIGS. 5A-5D: Innate immune response in primary T cells, in accordance with some embodiments. FIG. 5A: Schematic diagram of the GFP reporter cssDNA targeting RAB11A locus and TRAC locus (Upper). GFP reporter knock-in efficiency with cssDNA donor templates in human primary T cells. Immediately after electroporation, cells were treated with either DMSO or 1 μM M-3814 for 24 hours. The knock-in efficiency was examined on Day 7 post electroporation. FIG. 5B. Secreted IFN-γ and TNF-α cytokines levels in cultured primary T medium after electroporation with buffer, dsDNA, cssDNA or mRNA molecules. Conditioned media were collected for cytokine analysis using the Ella immunoassay platform with selected panels. FIGS. 5C-5D: Gene set enrichment analysis (GSEA) enrichment analysis of interferon gamma response genes on the differential expression genes (DEG) of from the dsDNA or cssDNA treated primary T cells. NES: normalized enrichment score; FDR q-value: False Discovery Rate-adjusted p-value. ****p<0.0001 One-Way ANOVA Bonferroni Post Hoc Test between indicated groups. “EP only” stands for electroporation only.

FIGS. 6A-6E: K562 genome engineering with paired spCas9 nickases and cssDNA. in accordance with some embodiments. FIG. 6A: Schematic diagram of sgRNA design for paired Cas9 nicking at exon 1 of RAB11A locus. Two PAM-out configurations were designed for the double Cas9 nicking (gRNA2+3 and gRNA2+3) with 57 bp and 41 bp apart between the nicking sites. FIG. 6B. GFP knock-in efficiency at RAB11A locus using cssDNA as donor template with paired Cas9 nicking design. Knock-in efficiency was examined on Day 7 post electroporation by flow cytometry. FIG. 6C. GFP flow cytometry data at RAB11A locus in K562 cells when Cas9 or D10ACas9 (nCas9) was delivered by mRNA. FIG. 6D. Quantification of the GFP knock-in efficiency when Cas9 or nCas9 was delivered by mRNA. FIG. 6E. Design of GFP reporter cssDNA donor template paired with Cpf1/gRNA nuclease editor at B2M locus and GFP knock-in efficiency at B2M locus using cssDNA donor template with Cpf1/gRNA. Knock-in efficiency was examined on Day 7 post electroporation by flow cytometry. **p<0.01, One-Way ANOVA Bonferroni Post Hoc Test between indicated groups.

FIGS. 7A-7I: CAR-T cell engineering with cssDNA, in accordance with some embodiments. FIG. 7A: Schematic diagram of nonviral CAR-T cell engineering process. Pan T cells are isolated from peripheral blood and activated on day 0 with anti-CD3/anti-CD28 TransAct. Cells are electroporated using the Lonza nucleofector on day 2 with Cas9 RNPs+cssDNA HDR donor templates and then expanded for a total of 7-10 days. FIG. 7B: Representative day 7 flow cytometry graphs showing CAR knock-in for Mock (un-engineered) and cssDNA-engineered T cells with DMSO or 1 μM M-3814 treatments. FIG. 7C: Representative day 7 flow plots and quantifications of CAR knock-in for mock (un-engineered), cssDNA- or AAV6-engineered T cells. Cells were treated with 1 μM M-3814 for 1 day immediately after electroporation. FIG. 7D: Expansion ability of engineered CAR-T cells. FIG. 7E: In vitro killing of NALM6 acute lymphoblastic leukemia cell line with cssDNA-, AAV6-engineered CAR-T cells in comparison to unmodified T cells from same blood donors after 24 hours of co-culture. The in vitro killing measured by live cell imaging using IncuCyte® live cell imaging system. FIG. 7F: The growth curve of target NALM6 cells when co-cultured with un-engineered, cssDNA- or AAV6-engineered CAR-T cells at various effector to target (E: T) ratios. FIG. 7G: the data of FIG. 7F, expressed as a bar chart. FIG. 7H, In vivo Xenograft study outline. NSG mice received 0.5×106 Luc-NALM-6 cells (i.v.) followed by 3×106 CAR+ T cells injection (i.v.). FIG. 7H, H. Kaplan-Meier analysis of the survival of tumor bearing NSG mice over a 21-day period after receiving CAR-T cell treatment. I. FIG. 7I, Tumor burden shown as bioluminescent signal quantified per animal every week over a 21-day period. n=5 mice per group.

FIGS. 8A-8E: CAR-NK cell engineering with cssDNA, in accordance with some embodiments. FIG. 8A, Schematic diagram of a nonviral NK cell engineering process. Primary NK cells were isolated from healthy donor's peripheral blood and expanded with EBV-LCL feeder cells for 12-16 days. Cells were then electroporated using the Lonza nucleofector with Cas9 RNPs+cssDNA HDR donor templates and then recovered/cultured for another 7-14 days. FIG. 8B, Fold expansion of primary NK cells when co-cultured with EBV-LCL feeder cells from either fresh isolation (Left) or recovered from cryopreservation (Right); C. Middle, schematic illustration of cssDNA donor template used for GFP reporter knock-in at RAB11A locus; Bottom, GFP reporter KI efficiencies (%) and cell viability of primary NK cells 7 days post engineering. Knock-in efficiency was determined by flow cytometry and cell viability was measured through NucleoCounter® NC-202™. D. Representative flow cytometry plots (7 days post engineering) showing GFP knock-in from control and cssDNA-engineered NK cells. FIG. 8D. Top, schematic illustration of cssDNA donor template used for 1928z-1xx CD19 CAR knock-in at RAB11A locus. Bottom, cell viability (%) of primary NK cells on indicated days after engineering, NK cell proliferation during 2 weeks after engineering, and in vitro cytotoxicity of engineered primary NK cells against target CD19+NALM6 cells. NK cells were co-cultured with target cells at different effector: target (E/T) ratios for 4 hours. Cytotoxicity was measured using Cell Counting Kit-8. FIG. 8E, Representative flow cytometry plots of CD19.CAR knock-in efficiency in NK cells 7 days after engineering. 1928z-1xx CD19.CAR was determined by staining with F(ab′) 2 Fragment Goat Anti-Mouse IgG antibodies.

FIG. 9: The stability of purified cssDNA under various storage conditions for as long as 14 days, or subjecting to freeze/thaw cycles, in accordance with some embodiments, cssDNA was aliquoted and stored at room temperature, 4° C. for up to 14 days (lanes 1-10) or was subjected to freeze-thaw cycles for up to 42 times over 14 days (lanes 11-16). An aliquot of cssDNA was stored at −20° C. for 14 days for comparison (lane 17). 100 ng of cssDNA was loaded into DNA agarose gel for imaging.

FIG. 10A-10B. Knock-in efficiency with cssDNAs harboring different lengths of homology arms, in accordance with some embodiments. FIG. 10A: Schematic diagram of GFP cssDNA targeting RAB11A locus flanked with different 5′ and 3′ homology arms. FIG. 10B: GFP knock-in efficiency with different cssDNA with various homology arm lengths. 500,000 K562 cells were co-electroporated with RAB11A RNP (spCas9 and sgRNA) and 1.8 μmol of cssDNA. Knock-in efficiency was determined by flow cytometry on Day 4 post electroporation.

FIGS. 11A-11B: Innate cellular immune response related gene expression level in human primary T cells, in accordance with some embodiments. Cultured human primary T cells were electroporated with buffer, dsDNA, cssDNA or mRNA. Cells were collected 24 hours post electroporation for RNA extraction and sequencing. Innate cellular immune response related gene panel was plotted for control (EP only), dsDNA, cssDNA or mRNA treated groups. FPKM, fragments per kilo base of transcript per million mapped fragments, ns, non-significant difference. *, p<0.05, ***, p<0.001, ****, p<0.0001 One-Way ANOVA Bonferroni Post Hoc Test between indicated groups.

FIGS. 12A-12B: Flow data confirming the expression of CD19 and CD22 in NALM6 cells, in accordance with some embodiments. NALM6 cells were stained with either APC anti-human CD19 Antibody (FIG. 12A) or PE anti-human CD22 Antibody (FIG. 12B) and subjected to flow analysis. Unstained control cells were used to determine background fluorescence.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of +20% or +10%, in certain embodiments+5%, in certain embodiments+1%, in certain embodiments+0.1% from the specified value, as such variations are appropriate to perform the disclosed methods.

Abbreviations: CAR-T: chimeric antigen receptor T cells. cssDNA: Circular single stranded DNA. gRNA: guide RNA. HDR: Homology directed repair. HR: Homologous recombination. KI: knock-in. sgRNA: Single guide RNA. ssDNA: Single stranded DNA.

### Method of Editing DNA

In one aspect, the study described herein (“the present study”) developed a circular single-stranded DNA donor template-based method of editing DNA, such as genomic DNA, in cells. In certain embodiments, the method achieved high editing efficiencies (i.e., the ratio between the number of edited cells to the total number of the cells) in various types of clinically relevant cell types, including pluripotent stem cells (iPSCs), hematopoietic stem cells (HSCs), B-cells, and T-cells. In certain embodiments, the method did not induce innate cellular immune response. In certain embodiments, cell viabilities during and after the DNA editing were high.

Accordingly, in some aspects, the present invention is directed to a method of editing a DNA in cells.

In some embodiments, the method comprises delivering into the cells a circular single-stranded DNA (cssDNA) donor template including a DNA insert; a 5′-homology arm; and a 3′-homology arm. In certain embodiments, the 5′ homology arm and the 3′ homology arm are complementary to the DNA in a target region.

In some embodiments, the method comprises delivering into the cells a DNA editing construct or a nucleic acid encoding the DNA editing construct. In certain embodiments, the DNA editing construct has a site-specific DNA endonuclease activity. In certain embodiments, the DNA editing construct introduces a DNA break in the target region of the DNA.

In some embodiments, the DNA insert of the cssDNA donor template is inserted into the target region via homologous recombination (HR) or homology directed repair (HDR), such as in response to the DNA break introduced by the DNA editing construct.

In some embodiments, the cssDNA donor template and/or the DNA editing construct is the same as or similar to those as described in WO 2020/142730, the entirety of which is hereby incorporated herein by reference.

The present study tested various parameters of the DNA editing method herein such as the amount of the cssDNA donor template, the waiting time, the inclusion/exclusion of additional nonhomologous end joining (NHEJ) inhibitors, etc., and discovered that high editing efficiencies can be achieved by the method herein even when payloads of 2,000 bases or larger were used. Accordingly, in some embodiments, a length of the DNA insert is about 2,000 bases or longer, and an efficiency of the editing is about 20% or higher, such as about 25% or higher, about 30% or higher, about 35% or higher, about 40% or higher, about 45% or higher, about 50% or higher, about 55% or higher, about 60% or higher, about 65% or higher, or about 70% or higher, based on a total number of the cells. In some embodiments, the length of the DNA insert is about 4,000 bases or longer, and the efficiency of the editing is about 10% or higher, such as about 15% or higher, about 20% or higher, about 25% or higher, about 30% or higher, or about 35% or higher, based on the total number of the cells. In some embodiments, the length of the DNA insert is about 8,000 bases or longer, and the efficiency of the editing is about 2% or higher, such as about 2.5% or higher, about 3% or higher, about 3.5% or higher, about 4% or higher, or about 5% or higher, based on the total number of cells. It is worth noting that the commonly used donor carrier AAV6 has a 4.5 Kb packaging limit. As such, the instant method is able to insert payloads longer than 8 Kb while still able to achieve reasonable editing efficiencies.

In some embodiments, the length of the DNA insert is less than about 2,000 bases, and an editing efficiency is about 30% or higher, such as about 35% or higher, about 40% or higher, about 45% or higher, about 50% or higher, about 55% or higher, about 60% or higher, about 65% or higher, about 70% or higher, about 75% or higher, about 80% or higher, based on the total number of the cells.

In some embodiments, the cells are induced pluripotent stem cells (iPSCs), hematopoietic stem cells (HSCs), B-cells, T-cells, or combinations thereof.

In some embodiments, the cells are T-cells, and the method is a method of preparing engineered T-cells, such as engineered T-cells having an altered T-cell receptor, or chimeric antigen receptor T cells (CAR-T cells).

In some embodiments, the cells are iPSCs, the length of the DNA insert is about 2,000 bases or longer, and the efficiency of the editing is 40% or higher based on a total number of the cells.

In some embodiments, the cells are hematopoietic stem cells (HSCs), the length of the DNA insert is about 2,000 bases or longer, and the efficiency of the editing is 40% or higher based on a total number of the cells.

In some embodiments, the cells are B-cells, the length of the DNA insert is about 2,000 bases or longer, and the efficiency of the editing is 20% or higher based on a total number of the cells.

In some embodiments, the cells are T-cells, the length of the DNA insert is about 2,000 bases or longer, and the efficiency of the editing is 40% or higher based on a total number of the cells.

In some embodiments, each of the 5′-homology arm and the 3′-homology arm independently has a length ranging from about 25 nucleotides to about 5000 nucleotides, such as from about 50 nucleotides to about 5000 nucleotides, from about 75 nucleotides to about 2500 nucleotides, from about 100 nucleotides to about 2000 nucleotides, from about 150 nucleotides to about 1500 nucleotides, from about 200 nucleotides to about 1000 nucleotides, or from about 300 nucleotides to about 500 nucleotides.

The DNA editing construct useful for the instant method is not limited. As long as the construct has a site-specific DNA endonuclease activity, i.e., the construct is able to induce a DNA break, such as a double strand DNA break or a single strand DNA break, at a specific site (as defined by nucleotide sequence) of a DNA molecule, the construct is considered a DNA editing construct herein.

In some embodiments, the DNA editing construct is or includes at least one site-specific nuclease selected from the group consisting of a Cas nuclease, a zinc-finger nuclease, a meganuclease, and a transcription activator-like effector nuclease (TALEN), including nickase forms thereof. A large number of site-specific DNA endonucleases are genetically engineered proteins, such as those described in Bogdanove et al. (Nucleic Acids Research, Volume 46, Issue 10, 1 Jun. 2018, Pages 4845-4871, the entirety of this reference is hereby incorporated herein by reference).

In some embodiments the DNA editing construct is or includes a class I Cas protein or a class II Cas protein, including nickase forms thereof. Class I and class II proteins are described in, for example, Makarova et al. (Nature Reviews Microbiology volume 18, pages67-83 (2020)). The entirety of this reference is hereby incorporated herein by reference.

In some embodiments, the DNA editing construct is or comprises Cas9, Cas3, Cas10, Cas11, CasX, Cas12a, MAD7, including nickase forms thereof.

In some embodiments, the DNA is a genomic DNA.

The present study discovered that the method herein is able to introduce biallelic edit in a significant percentage of edited diploid cells. Accordingly, in some embodiments, the cells include two or more copies of the DNA, and wherein about 20% or more, such as about 25% or more, about 30% or more, about 40% or more, 50% or more, or 60% or more, of the edited cells are edited in two or more copies of the DNA.

The delivery method of the construct, the nucleic acid encoding the construct, and/or the ssDNA into the cells are not limited. Since the methods of delivering proteins or nucleotides into cells are well known in the art, the delivery step is not detailed herein. Non-limiting examples of means of delivery includes viral vectors, lipid or non-lipid nanoparticle deliveries, exosome deliveries, electroporation deliveries, gene gun deliveries, injections, or the like.

The present study discovered that nonhomologous end joining (NHEJ) inhibitors, such as DNA-dependent protein kinase (DNA-PK) inhibitors M-3814 and Alt-R HDR enhancer 2 were able to significantly increase the editing efficiency when used in conjunction with the cssDNA based editing method herein.

Accordingly, in some embodiments, contacting the cells with an NHEJ inhibitor. In some embodiments, the NHEJ inhibitor is a DNA-PK inhibitor. In some embodiments, the DNA-PK includes M-3814 or Alt-R HDR enhancer 2.

### Kit for Editing DNA

The present study discovered that the combined effects of cssDNA donor template, DNA editing constructs herein, and nonhomologous end joining (NHEJ) inhibitors are able to achieve high editing efficiencies in various cell types.

Accordingly, in some aspects, the present invention is directed to a kit for editing DNA.

In some embodiments, the kit includes a circular single-stranded DNA (cssDNA) donor template including a DNA insert; a 5′-homology arm; and a 3′-homology arm. In some embodiments, the 5′ homology arm and the 3′ homology arm are complementary to the DNA in a target region;

In some embodiments, the kit further includes a DNA editing construct or a nucleic acid encoding the DNA editing construct. In some embodiments, the DNA editing construct has a site-specific DNA endonuclease activity. In some embodiments, the DNA editing construct introduces a DNA break in the target region of the DNA.

In some embodiments, the kit further includes a nonhomologous end joining (NHEJ) inhibitor or a DNA recombinase stimulator.

In some embodiments, the cssDNA donor template, the DNA editing construct, the nucleic acid encoding the DNA editing construct, and/or the NHEJ inhibitor are the same as or similar to those described elsewhere herein, such as in the “Method of Editing DNA” section.

In some embodiments, the kit further includes a component for delivering the DNA editing construct, the nucleic acid encoding the DNA editing construct, and/or the ssDNA into a cell. In some embodiments, the component includes a microinjector, a lipid for forming lipid nanoparticles, an exosome, a gene gun, a metal nanoparticle (such as a gold or tungsten nanoparticle), a cuvette for electroporation, or the like.

### Method of Killing a Cancer Cell

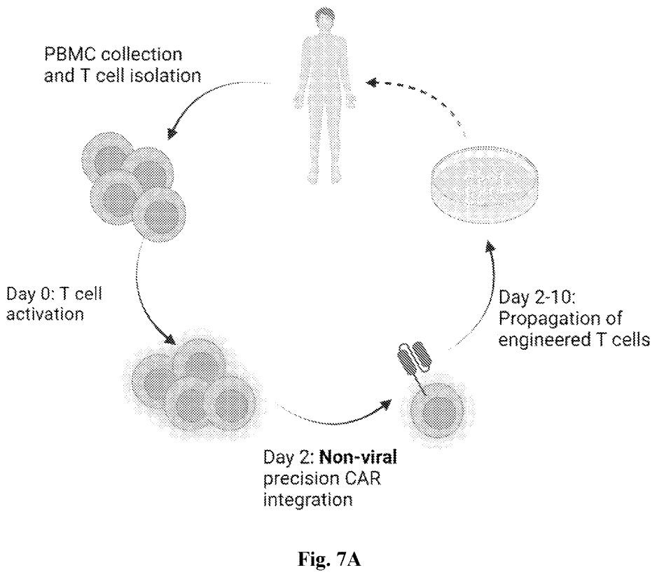

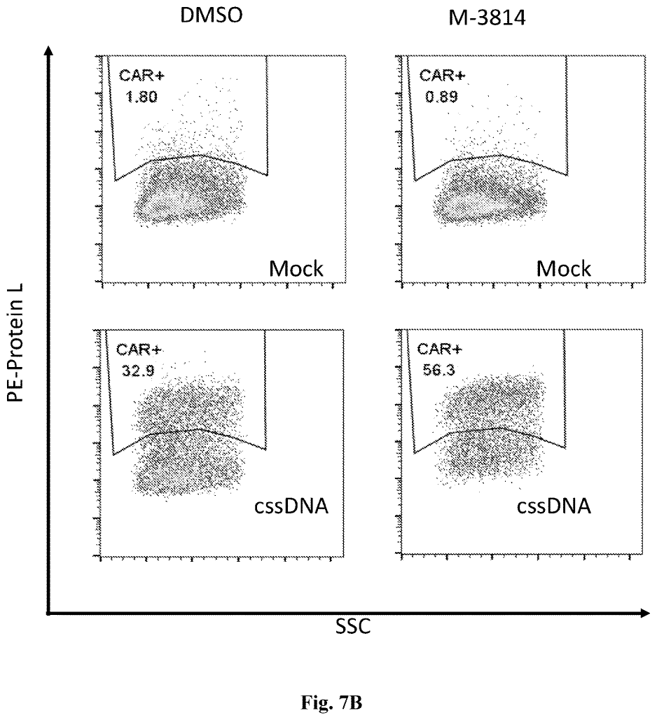

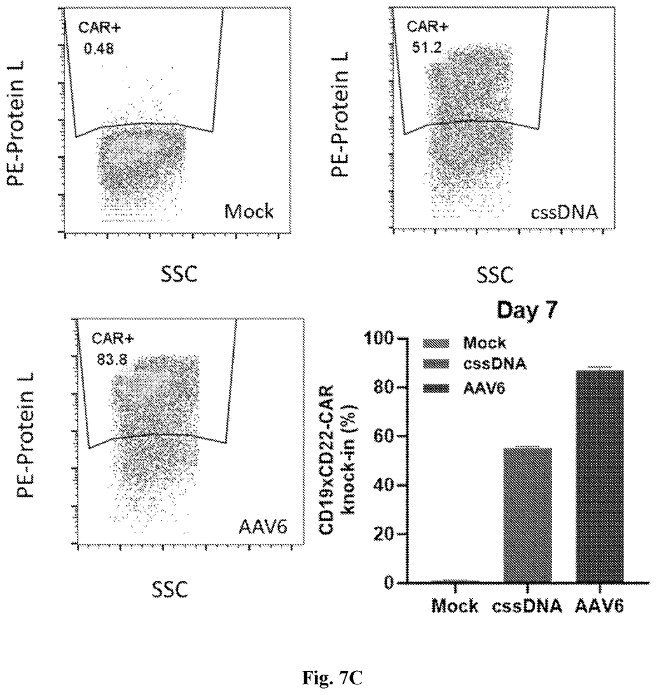

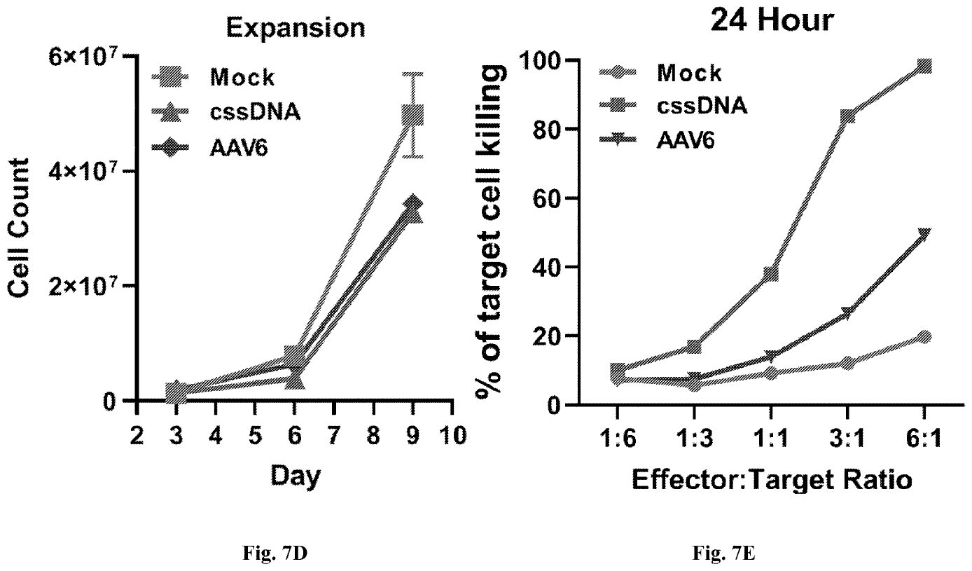

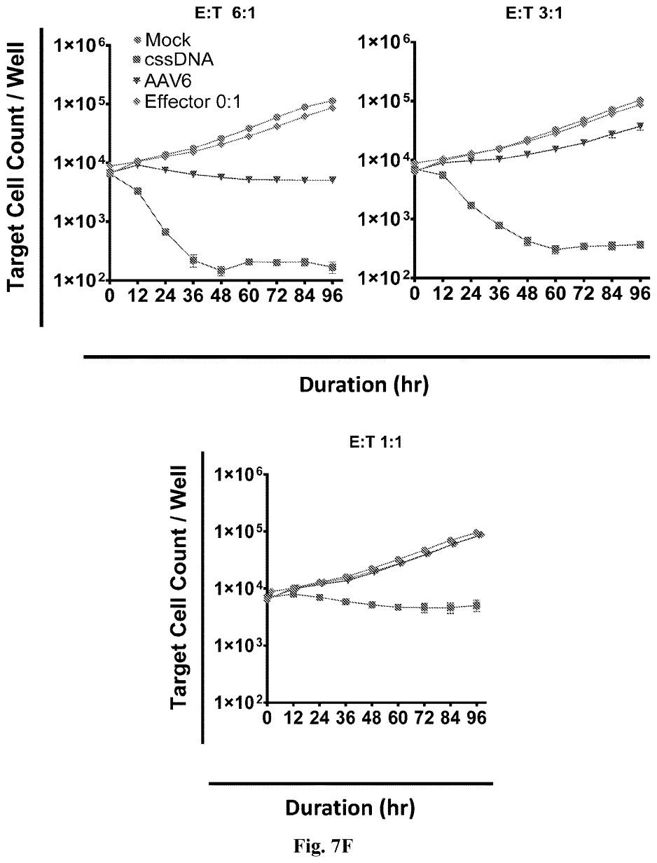

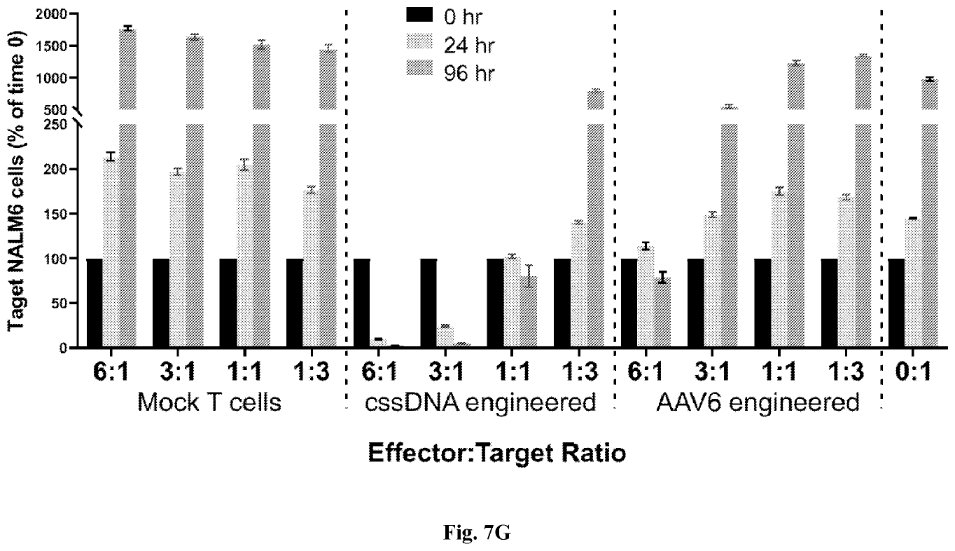

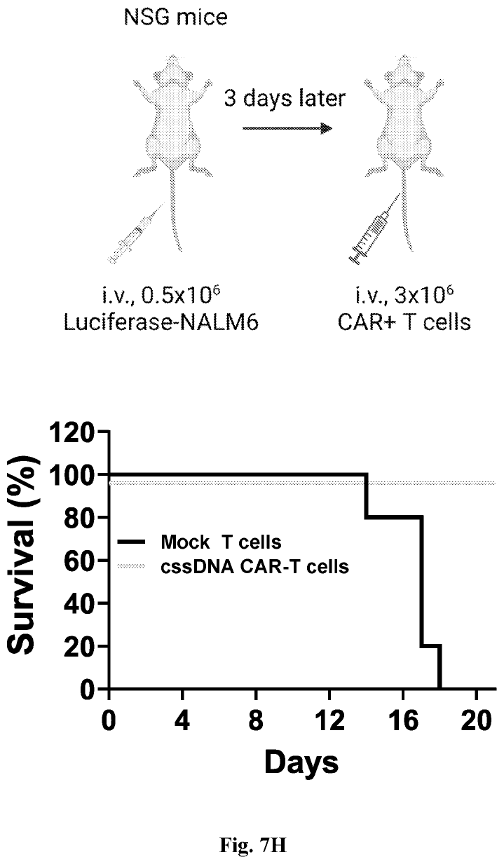

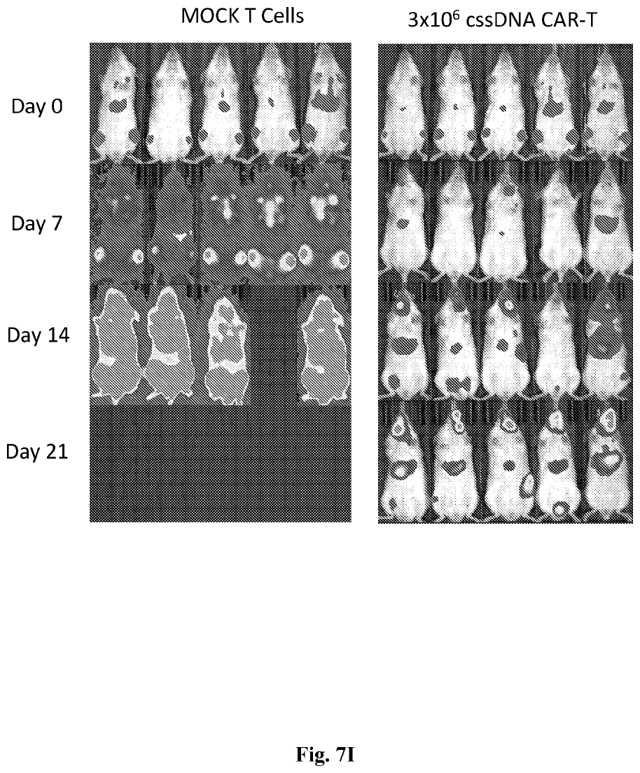

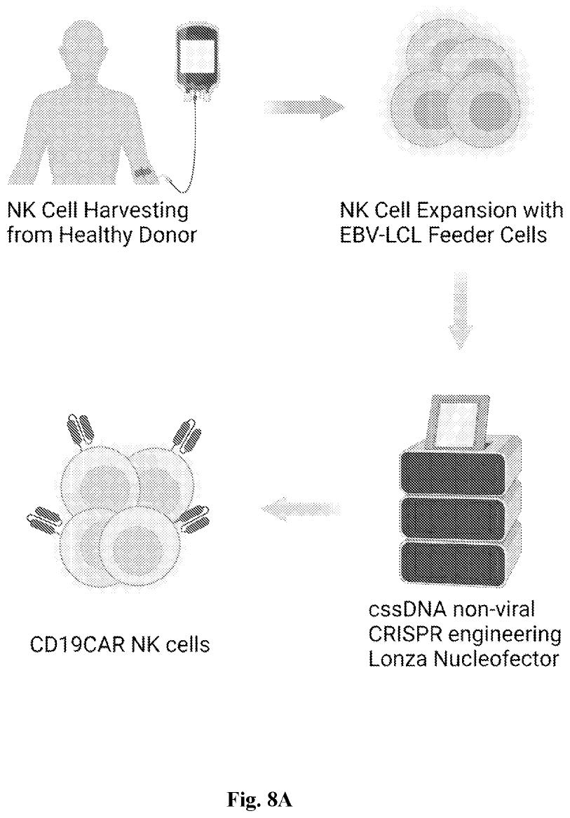

The present study prepared engineered T-cells, such as chimeric antigen receptor T-cells (CAR-T cells), by editing T-cells using the DNA editing method developed in the study. Unexpectedly, although the editing efficiency of the method herein is lower than that achieved by the conventional AAV6-based editing method, the engineered T-cells engineered using the method herein were shown to have much stronger cancer-killing potency than the comparable engineered T-cells engineered using the AAV6-based method (see e.g., FIGS. 7A-7G).

Accordingly, in some aspects, the present invention is directed to a method of killing a cancer cell.

In some embodiments, the method includes contacting the cancer cell with engineered T-cell including an engineered antigen receptor (such as engineered T-cell receptor (TCR) or a chimeric antigen receptor (CAR)) targeting the cancer cell.

In some embodiments, the engineered T-cell is an engineered T-cell, including an engineered TCR. In some embodiments, the engineered T-cell is a CAR-T cell.

In some embodiments, the engineered T-cell cell is prepared by delivering into a T-cell a circular single-stranded DNA (cssDNA) donor template including a DNA insert; a 5′-homology arm; and a 3′-homology arm. In certain embodiments, the 5′ homology arm and the 3′ homology arm are complementary to the DNA in a target region.

In some embodiments, the engineered T-cell cell is prepared by delivering into a T-cell a DNA editing construct or a nucleic acid encoding the DNA editing construct. In certain embodiments, the DNA editing construct has a site-specific DNA endonuclease activity. In certain embodiments, the DNA editing construct introduces a DNA break in the target region of the DNA.

In some embodiments, when preparing the engineered T-cell cell from the T-cell, the DNA insert is inserted into the target region via homologous recombination (HR), or homology directed repair (HDR).

In some embodiments, the cssDNA donor template, the DNA editing construct, and/or the nucleic acid encoding the DNA editing construct are the same as or similar to those described herein, such as in the “Method of Editing DNA” section.

In some embodiments, the method of killing the cancer cell further includes the step of preparing the engineered T-cell cell. In some embodiments, the step of preparing the CAR-T cell is not part of the method of killing the cancer cell.

Without wishing to be bound by theory, it is hypothesized that the superior cancer killing results of the engineered T-cells engineered by the instant cssDNA based editing methods are results of low cytotoxicity of the cssDNA method (thus healthier engineered T-cells), and/or the ability of the method to introduce significant level of biallelic editing (thus higher dosage of the engineered antigen receptors expressed by the engineered T-cells). The nature of the antigen receptors engineered into the T-cells per se is not important and any antigen receptors that have been shown to be effective when engineered using other methods, such as viral vector based methods, are expected to be useful for the method herein. In some embodiments, the insertion of the DNA insert produces a chimeric antigen receptor targeting at least one selected from the group consisting of B-cell maturation antigen (BCMA), carcinoembryonic antigen (CEA), CD2, CD4, CD5, CD7, CD19, CD22, CD30, claudin 18.2 (CLDN 18.2), epithelial cellular adhesion molecule (EpCAM), folate receptor 1 (FOLR1), human epidermal growth factor receptor 2 (HER2), mesothelin (MSLN), mucin 1 (MUC1), natural killer group 2D (NKG2D). Chimeric antigen receptors are also described in, for example, Chu et al. (Contemp Oncol (Pozn). 2018 March; 22(1A): 73-80). The entirety of the reference is hereby incorporated herein by reference. In some embodiments, the insertion of the DNA insert produces an engineered TCR that has altered antigen specificity compared with the original T-cells.

In some embodiments, the target region of the DNA in which the DNA insert is inserted is a B2M locus, a genomic safe harbor locus (such as AAVS1 locus, hRosa26, Rogi1, Rogi2, GSH1-6 and/or a CCR5 locus), a PD1 locus, a TRAC locus, or combinations thereof. Safe harbor loci suitable for genetic engineering or for CAR engineering are described in, for example, Aznauryan et al. (Cell Rep Methods. 2022 Jan. 14; 2 (1): 100154) and Odak et al. (Blood, Volume 136, Supplement 1, 5 Nov. 2020, Page 28). Both of the references are hereby incorporated herein by reference.

In some embodiments, the cancer cell is a leukemia cancer cell, such as but not limited to an acute lymphoblastic leukemia cancer cell or a B-cell non-Hodgkin lymphoma cancer cell.

In some embodiments, the cancer cell is a cell in a cancer cell line, a primary cancer cell, or a cancer cell in the body of a subject.

In some embodiments, the cancer cell is in the body of the subject, and wherein the subject is a mammal, such as a human.

Method of Treating, Ameliorating, and/or Preventing Cancer

The present study prepared engineered T-cells (such as chimeric antigen receptor T-cells (CAR-T cells)) by editing T-cells using the DNA editing method developed in the study. Unexpectedly, although the editing efficiency of the method herein is lower than that achieved by the conventional AAV6-based editing method, the engineered T-cells engineered using the method herein were shown to have much stronger cancer-killing potency than the comparable engineered T-cells engineered using the AAV6-based method (see e.g., FIGS. 7A-7G).

Accordingly, in some aspects, the present invention is directed to a method of treating, ameliorating and/or preventing cancer, such as in a subject in need thereof.

In some embodiments, the method includes administering to the subject an effective amount of engineered T-cells including an engineered antigen receptor (such as engineered T-cell receptor (TCR) or a chimeric antigen receptor (CAR)) targeting the cancer cell.

In some embodiments, the engineered T-cells are engineered T-cells including an engineered TCR. In some embodiments, the engineered T-cells are CAR-T cells.

In some embodiments, the engineered T-cells are prepared by delivering into T-cells a circular single-stranded DNA (cssDNA) donor template including a DNA insert; a 5′-homology arm; and a 3′-homology arm. In some embodiments, the 5′ homology arm and the 3′ homology arm are complementary to the DNA in a target region.

In some embodiments, the engineered T-cells are prepared by delivering into a T-cell a DNA editing construct or a nucleic acid encoding the DNA editing construct. In some embodiments, the DNA editing construct has a site-specific DNA endonuclease activity. In some embodiments, the DNA editing construct introduces a DNA break in the target region of the DNA.

In some embodiments, when preparing the engineered T-cells from the T-cells, the DNA insert is inserted into the target region via homologous recombination (HR), or homology directed repair (HDR).

In some embodiments, the cssDNA donor template, the DNA editing construct, and/or the nucleic acid encoding the DNA editing construct are the same as or similar to those described herein, such as in the “Method of Editing DNA” section.

In some embodiments, the method of treating, ameliorating and/or preventing cancer further includes the step of preparing the CAR-T cell. In some embodiments, the step of preparing the CAR-T cell is not part of the method of treating, ameliorating and/or preventing cancer.

In some embodiments, the insertion of the DNA insert produces a chimeric antigen receptor targeting at least one selected from the group consisting of B-cell maturation antigen (BCMA), carcinoembryonic antigen (CEA), CD2, CD4, CD5, CD7, CD19, CD22, CD30, claudin 18.2 (CLDN 18.2), epithelial cellular adhesion molecule (EpCAM), folate receptor 1 (FOLR1), human epidermal growth factor receptor 2 (HER2), mesothelin (MSLN), mucin 1 (MUC1), natural killer group 2D (NKG2D), or other chimeric antigen receptors reported in the art.

In some embodiments, the target region of the DNA in which the DNA insert is inserted is a B2M locus, a genomic safe harbor locus (such as AAVS1 locus, hRosa26, Rogi1, Rogi2, GSH1-6 and/or a CCR5 locus), a PD1 locus, a TRAC locus, or combinations thereof.

In some embodiments, the cancer is a leukemia cancer, such as acute lymphoblastic leukemia cancer or B-cell non-Hodgkin lymphoma.

In some embodiments, the subject is a mammal, such as a human.

### EXAMPLES

The instant specification further describes in detail by reference to the following experimental examples. These examples are provided for purposes of illustration only and are not intended to be limiting unless so specified. Thus, the instant specification should in no way be construed as being limited to the following examples, but rather, should be construed to encompass any and all variations which become evident as a result of the teaching provided herein.

### Example 1: Circular Single-Stranded DNA is a Superior Homology-Directed Repair Donor Template for Efficient Genome Engineering

The toolbox for genome editing in basic research and therapeutic applications is rapidly expanding. While efficient targeted gene ablation using nuclease editors has been demonstrated from bench to bedside, precise transgene integration remains a technical challenge. AAV6 has been a prevalent donor carrier for homology-directed repair (HDR) mediated genome engineering but has reported safety issues, manufacturing constraints, and restricted applications due to its 4.5 Kb packaging limit. Non-viral targeted genetic knock-ins rely primarily on double-stranded DNA (dsDNA) donors and linear single-stranded DNA (lssDNA) donors. Both dsDNA and lssDNA have been previously demonstrated to have low efficiency and cytotoxicity. Here, the present invention developed a non-viral genome writing catalyst (GATALYST) system which allows production of ultrapure, minicircle single-stranded DNAs (cssDNAs) up to ˜20 Kb as donor templates for highly efficient precision transgene integration. GATALYST cssDNA donors enable knock-in efficiency of up to 70% in induced pluripotent stem cells (iPSCs), superior efficiency in multiple clinically relevant primary cell types, and at multiple genomic loci implicated for clinical applications with various nuclease editor systems. When applied to immune cell engineering, GATALYST cssDNA engineered CAR-T cells exhibit more potent and durable anti-tumor efficacy than those engineered from AAV6 viral vectors. The exceptional precision and efficiency, improved safety, payload flexibility, and scalable manufacturability of GATALYST unlocks the full potential of genome engineering with broad applications in therapeutic development, disease modeling and other research areas.

### Example 2

The RNA-guided Cas9 nucleases from the microbial CRISPR (clustered regularly interspaced short palindromic repeat)-Cas systems (CRISPR-Cas9) are robust and versatile tools for targeted genome editing tools in eukaryotic cells. CRISPR-Cas9 offers unprecedented opportunities to modify genome sequences in primary human cells throughout the field of cell and gene therapies. Cas nucleases are directed to a region in the genome by a guide RNA (gRNA) and induce a targeted double-strand break (DSB), where the resulting cellular repair mechanisms can be exploited to induce either error-prone or defined alterations. In the absence of a repair template, the cell typically repairs the lesion by a non-templated repair pathway such as nonhomologous end joining (NHEJ). In the presence of a homology-directed repair (HDR) DNA template, precise repair directed by regions of homology on DNA donor templates is triggered.

Previously, large disease modifying transgenes were introduced to various cell types via lentivirus or retrovirus. These methods harbored inherent challenges which led to technical challenges, resulting in undesirable phenotypes, like cancer. Such challenges include hard to track insertion genomic locus and copy number, difficulty manufacturing quality viral particles, and intrinsic heterogeneity of engineered cells. Recently, targeted transgene insertion has been successfully demonstrated in the chimeric antigen receptor T cells (CAR-T) field using DNA donor template encapsulated in recombinant associated-Adenovirus (rAAV). However, in addition to the potential viral-related safety and manufacturing challenges, the donor template payload application of rAAV is restricted by the packaging limit of 4.5 Kb.

Non-viral targeted genome editing, especially large transgene insertion, represents a significant unmet need, technically and medically, in the field of immune oncology and monogenic disorders. Non-viral targeted gene knock-in usually relies on double-strand DNA (dsDNA) donors, either linear or circular, to achieve targeted DNA insertion at a locus of interest. However, the use of dsDNA suffers from low efficiency and high cytotoxicity. More recently, single-stranded DNAs (ssDNAs) have been proven more effective than dsDNAs templates as donors for HDR in CRISPR-based genome editing due to reduced cellular toxicity and increased HDR efficiency. Moreover, illegitimate random integration is expected to be less frequent for ssDNA compared to dsDNA templates. ssDNA has become a groundbreaking tool for a wide variety of applications including engineering of human primary T cells. An endogenous T cell receptor (TCR) replaced with NY-ESO-1 antigen-specific 1G4 TCR encoded in ˜2 Kb long ssDNA demonstrated superior therapeutic efficacy compared to traditional methods involving TCR insertion using lentivirus. In addition, a hybrid ssDNA HDR template encoding an anti BCMA CAR targeting TRAC locus in primary T cells revealed superior cellular immunophenotype and rapid in vivo tumor clearance. The ssDNA donors used in these seminal studies were exclusively linear molecules produced with in vitro methods. They were produced by manipulation of DNA with polymerase chain reaction (PCR) coupled with enzymatic degradation of one of two DNA strands. However, the enzymatic production of linear ssDNA (lssDNA) templates is inherently inefficient and only commercially available at up to 5 Kb, due to accumulation of mutations from polymerase chain reaction, expensive reagent cost, and low scalability, thus prohibiting successful clinical application.

Phagemid-derived long circular ssDNA (cssDNA) HDR donors have higher efficiency of HDR-based repair in multiple cell lines (HEK293T and K562 cells). The cssDNA HDR templates outperform other forms of DNA donor due to its increased HDR efficiency, high specificity, large length capacity, and low cytotoxicity.

Here, the present study developed a technology platform using cssDNA purified from engineered phagemids for targeted gene writing. The present study established that cssDNA donor templates enable higher knock-in efficiency with decreased cytotoxicity when compared to lssDNA or dsDNA in cell lines, iPSC cells, and clinically relevant primary cells, allowing its potential applications for genetic disorders and immune cell therapy.

### Example 3: Production of cssDNA in a Highly Engineered Phagemid System

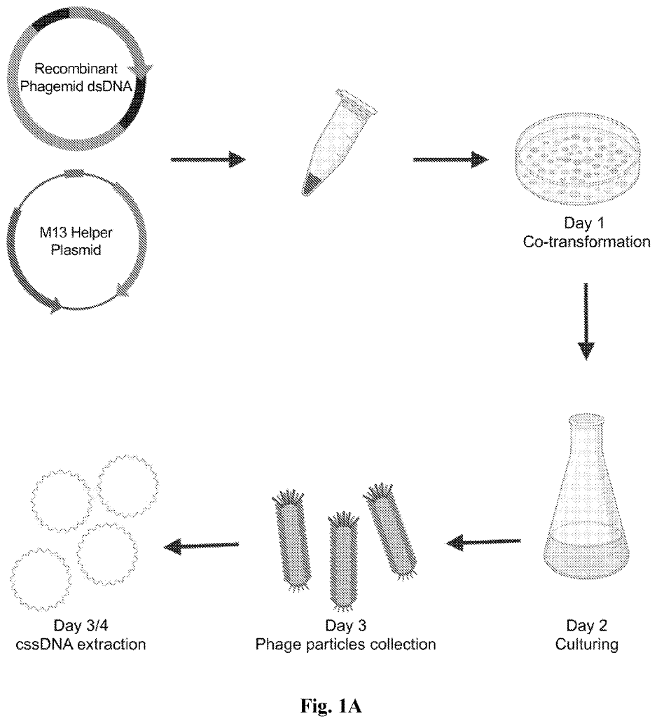

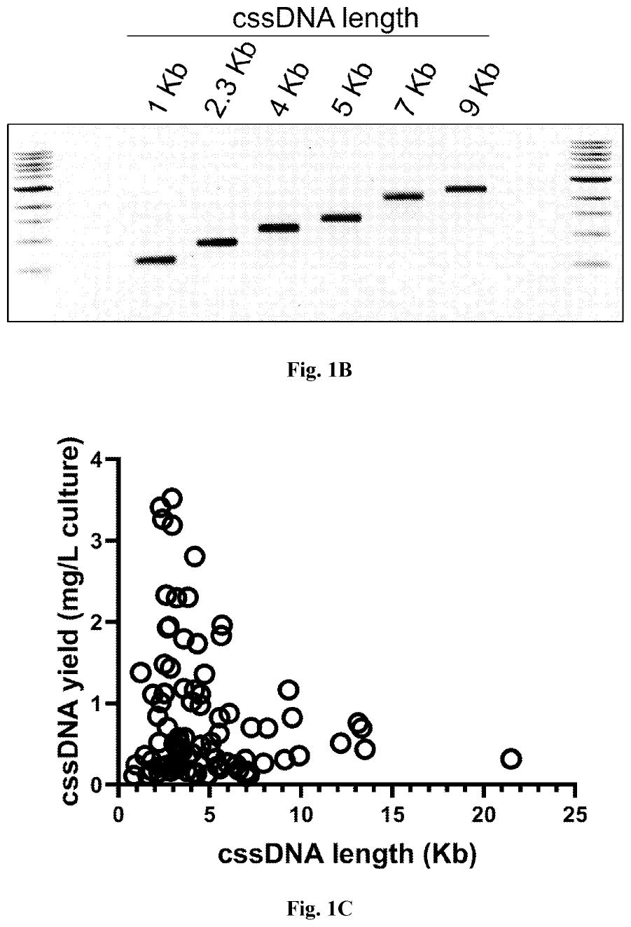

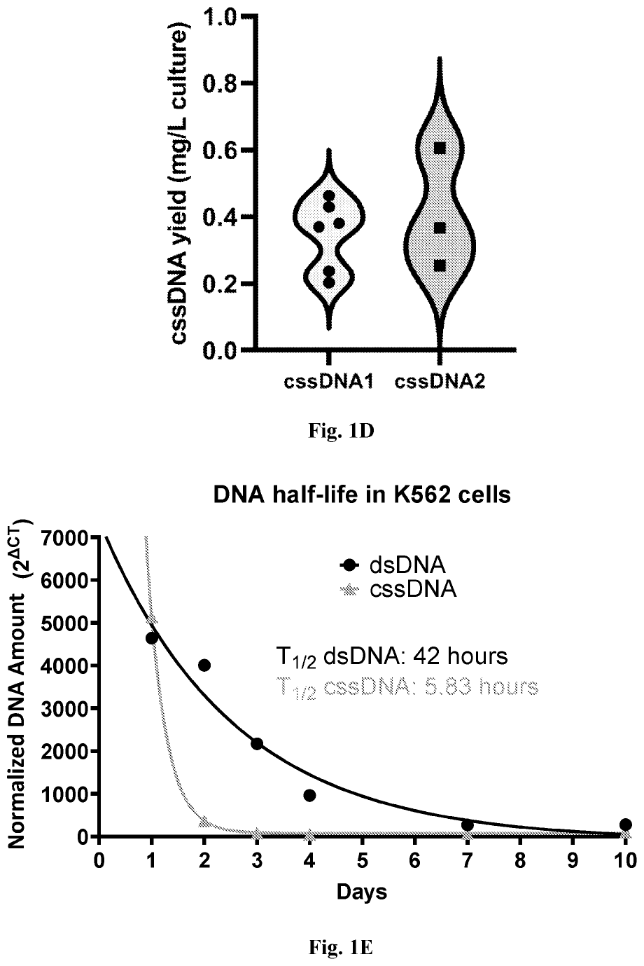

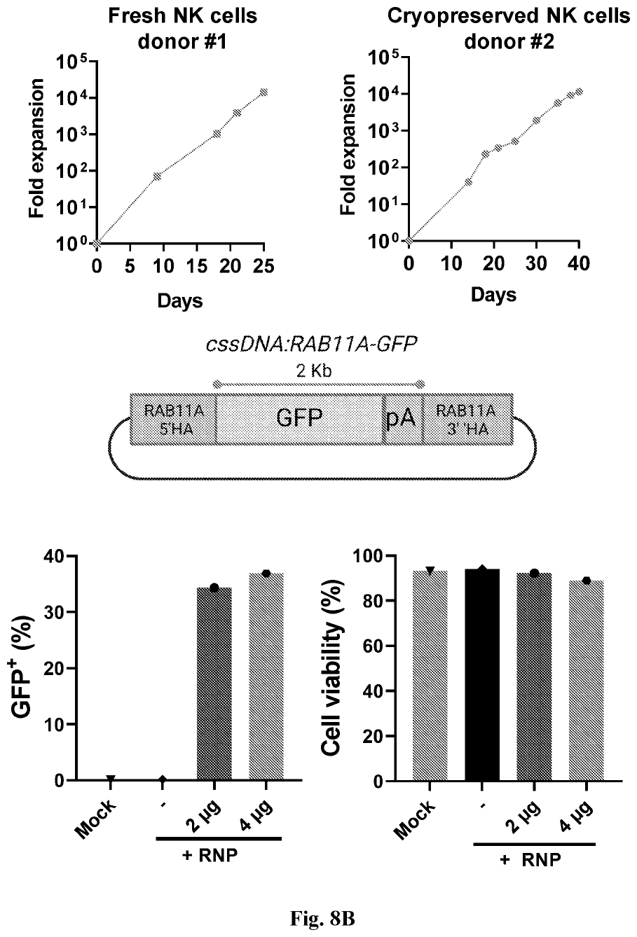

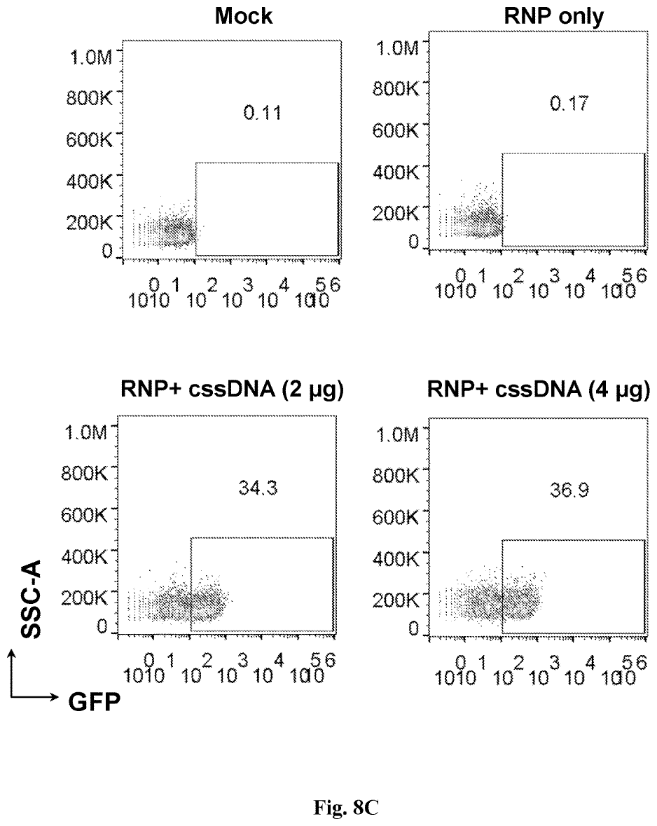

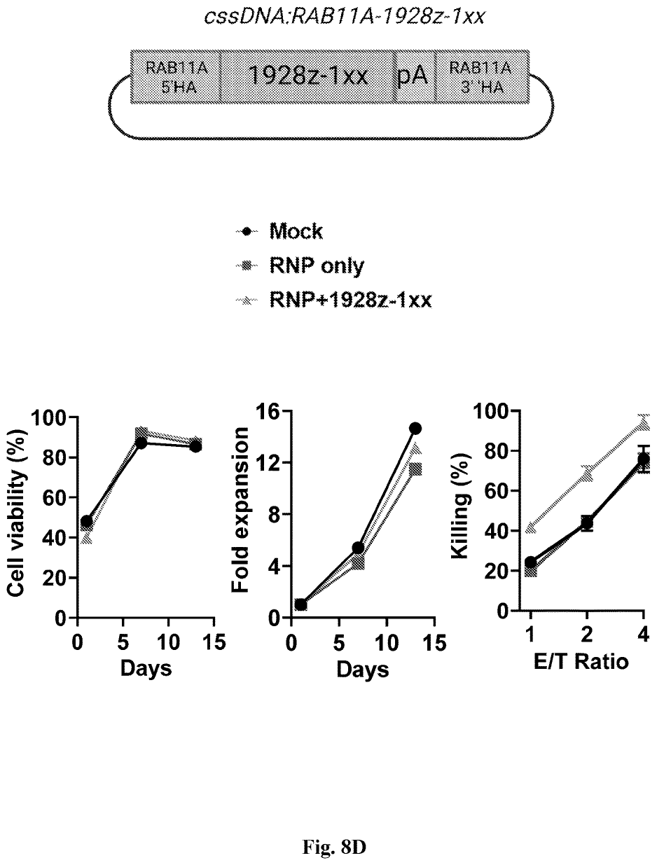

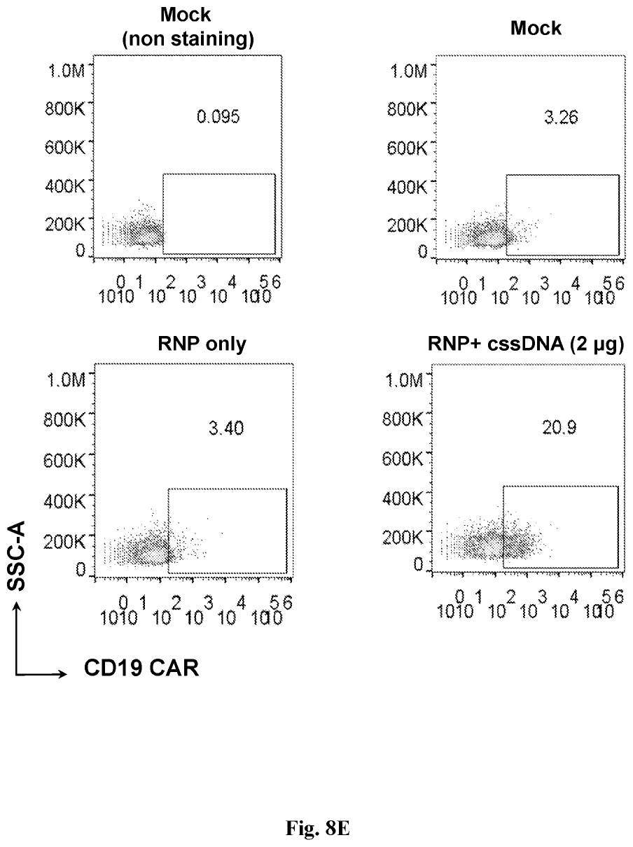

The present study developed a proprietary M13 phagemid system that allows the production of cssDNA. After co-transformation with a helper plasmid in XL1-blue E. Coli, the single strand of the phagemid vector is packaged into phage particles and extruded into the culture media (FIG. 1A). The endotoxin-free cssDNA extracted phage particles were then isolated using anion exchange chromatography column. Over 100 cssDNAs ranging from 1 Kb to 22 Kb in lengths were purified using this engineered phagemid system. Agarose gel imaging showed over 95% cssDNA purity across a range of lengths (FIG. 1B). There was no negative correlation between the yield and the length of the cssDNA (FIG. 1C). Thus, the yield of cssDNA is not expected to be compromised for larger cssDNA. Up to 22 Kb cssDNA can be readily generated. A varying yield was observed across different cssDNA constructs, though. Moreover, the cssDNA yield from multiple production batches for the same cssDNA molecule was consistent (FIG. 1D), suggesting the production process is reproducible and easily scalable. ssDNA structures are highly flexible, and their functional form is thermodynamically less stable. The circular form of ssDNA derived from phagemid is considered more resistant to exonuclease than lssDNA. The purified cssDNA was very stable and did not degrade after 14-days of storage at room temperature and withstood up to 42 freeze/thaw cycles over this period (FIG. 8). To determine the stability of cssDNA in a cellular context, a qPCR method was developed to quantify the amount of dsDNA or cssDNA from the total DNA extracted from cell lysate. Once dsDNA or cssDNA was delivered into mammalian K562 cells by electroporation, cell pellets were collected at different time points and the amount of dsDNA or cssDNA were quantified by qPCR. The half-life of the circular phagemid dsDNA in the cells was much longer than cssDNA with half-life ˜42 hours vs. 5.8 hours (FIG. 1E). This demonstrates that cssDNA is indeed less stable than dsDNA. However, this shorter half-life of cssDNA in cells could be advantageous for genome editing to minimize the undesired cellular toxicity and recombination.

### Example 4: GATALYST™ Gene Writing Platform with cssDNA Outperforms Other Non-Viral Nucleic Acid Payloads

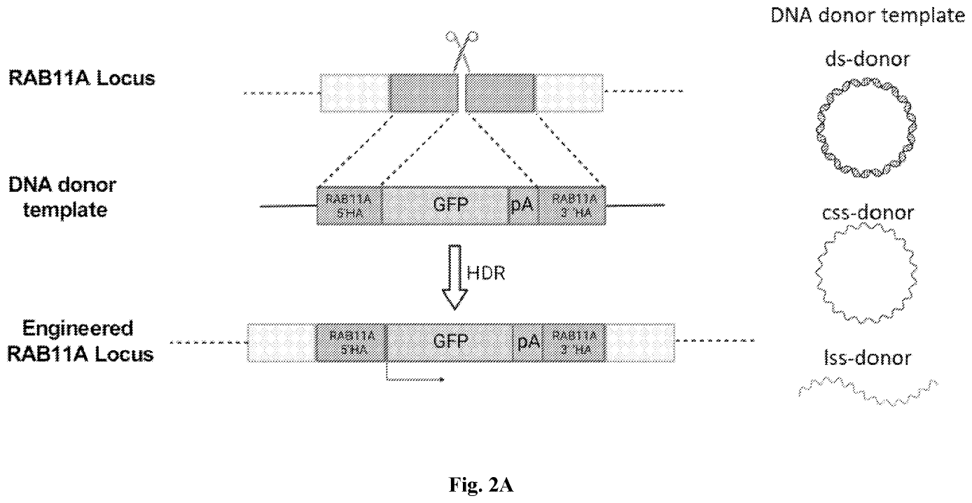

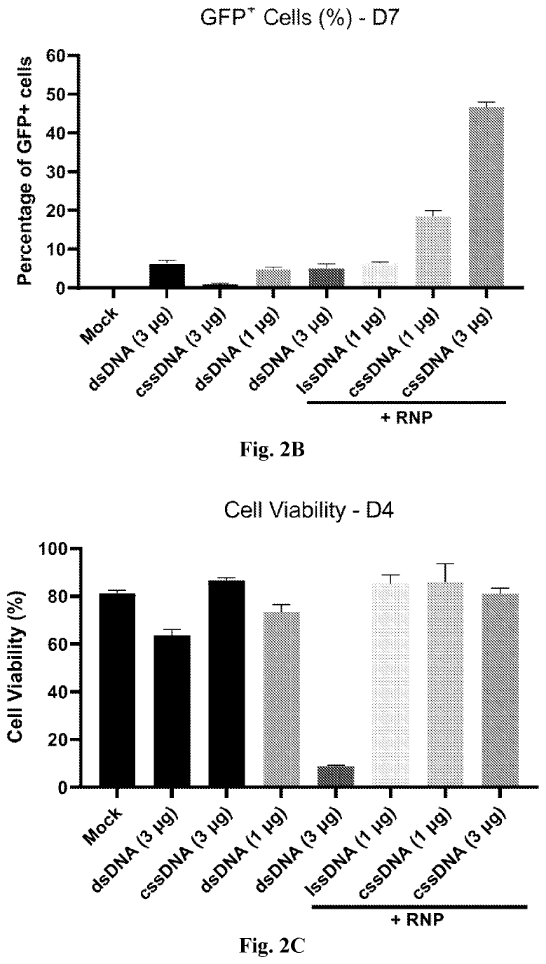

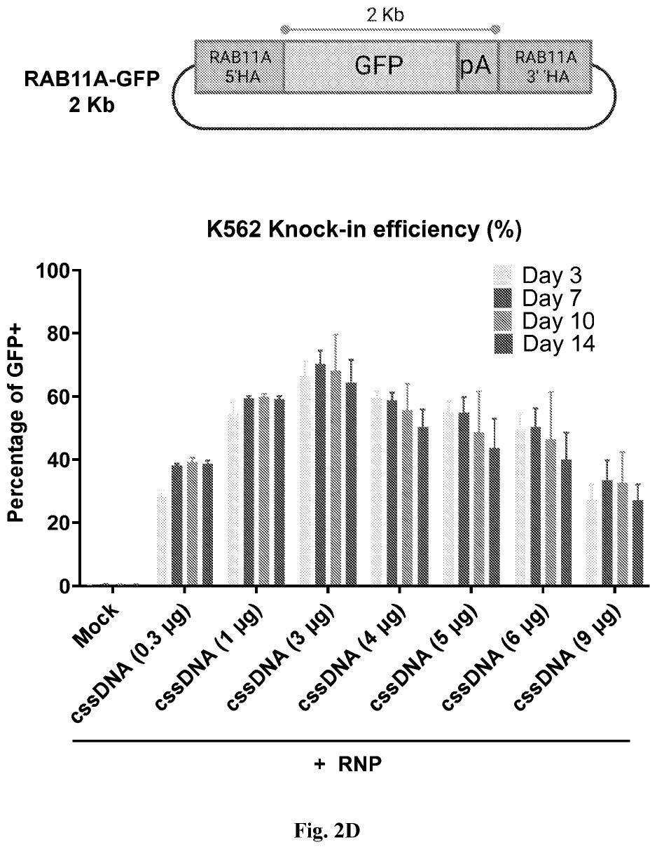

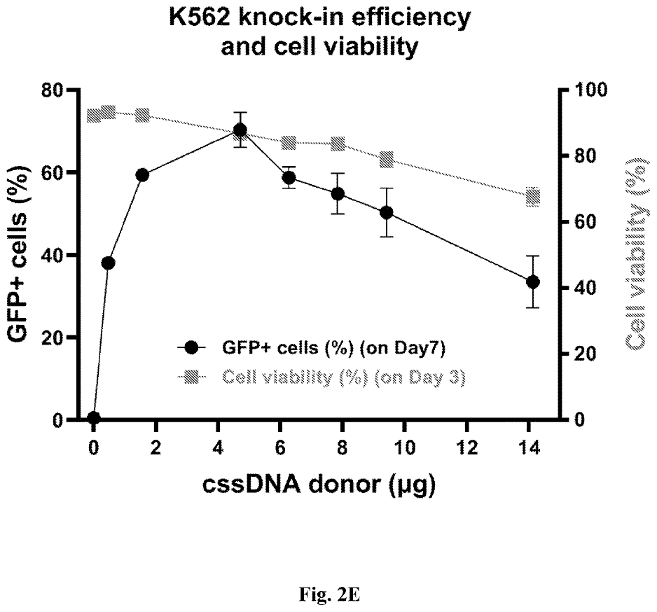

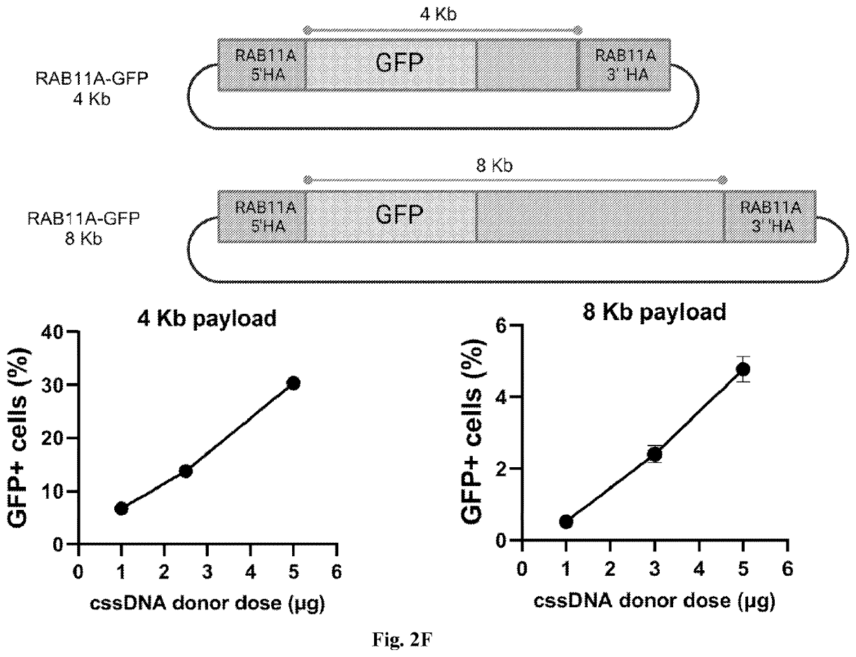

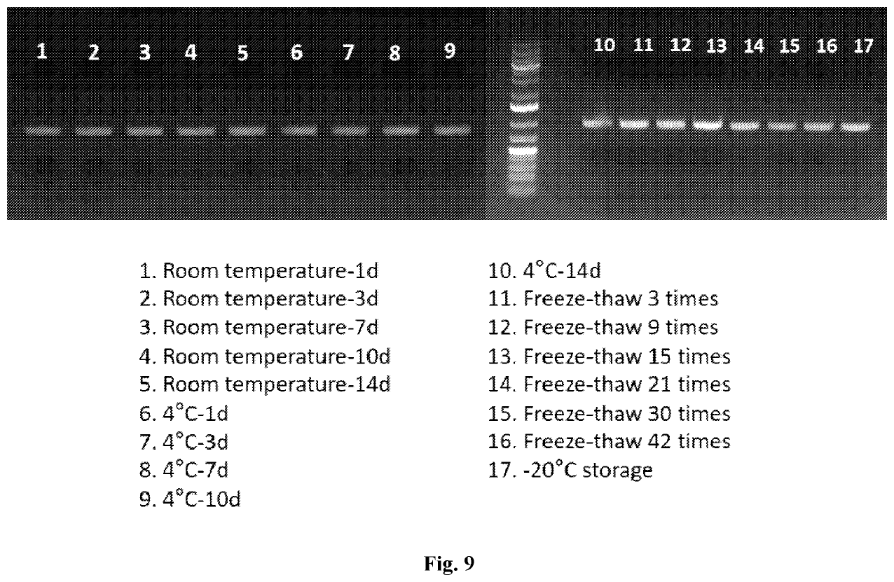

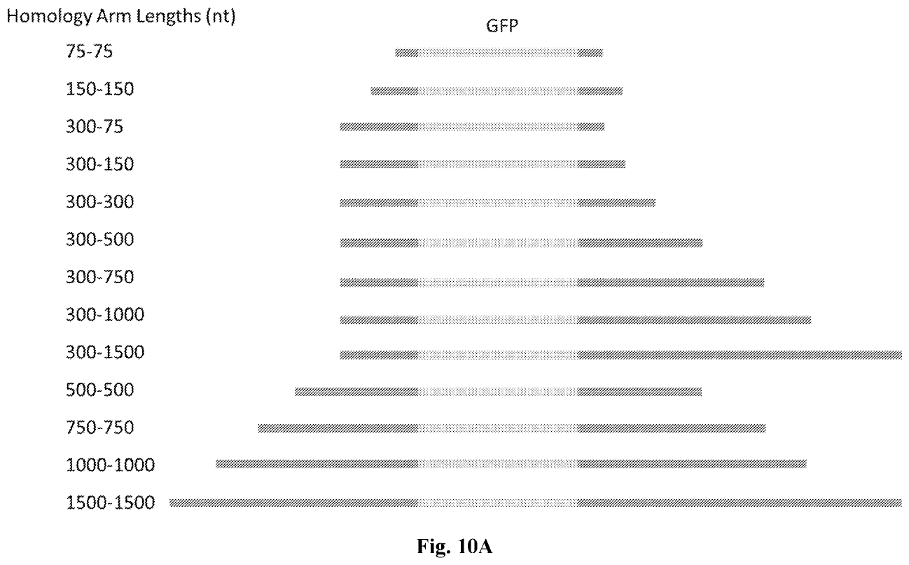

To directly compare the knock-in efficiency with different types of donor template, GFP reporter DNA donor templates flanked with ˜300 nt 5′ homology arm and ˜300 nt 3′ homology arm targeting RAB11A locus in the form of double-stranded circular plasmid, linear single-stranded or circular single-stranded were tested in K562 cells. As illustrated in FIG. 2A, when DNA donor templates were co-electroporated with ribonucleoprotein (RNP) complex consisting of spCas9 protein and single guide RNA (sgRNA) targeting exon 1 of RAB11A locus, precise HDR of the locus was evaluated by GFP reporter expression using flow cytometry. Day 7 post electroporation, knock-in efficiency as measured by GFP⁺ cell percentage was much higher with cssDNA as repair donor template, reaching to over 40% with 3 μg of cssDNA (FIG. 2B). In contrast, reduced knock-in efficiency was observed for lssDNA or dsDNA donor template. As expected, cell health was not compromised by cssDNA or lssDNA; however, the cell viability of 3 μg dsDNA engineered cells was less than 10% (FIG. 2C). Higher knock-in efficiency and lower cell toxicity suggests cssDNA is a superior donor DNA template over lssDNA and dsDNA for HDR-mediated genome knock-in. To examine this further, the present study then tested the cssDNA knock-in efficiency at various timepoints post electroporation across a range of dosages. FIG. 2D showed that cssDNA-mediated knock-in at RAB11A locus plateaued on Day 7 and sustained for at least 2 weeks. When knock-in efficiency (examined on Day 7) was plotted together with cell viability (examined on Day 3 post electroporation), a clear dose-dependent increase of knock-in efficiency for up to 5 μg of cssDNA, emerged, all while maintaining more than 80% cell viability. With higher concentrations of cssDNA (to 14 μg) was used, both knock-in efficiency and cell viability reduced (FIG. 2E). Thus, maximal knock-in efficiency is achieved with ˜5 μg cssDNA. The present study tested editing efficiency for cssDNA donor templates targeting RAB11A locus with different lengths of the 5′ and 3′ homology arms (FIG. 9A). Knock-in efficiency data indicated that 300 nt homology arm of each end resulted in the highest HDR efficiency, and there is no additional benefit when increasing the lengths up to 1500 nt (FIG. 9B). Thus, HDR cssDNA donor templates with 300 nt homology arms lengths were used in other genome engineering experiments in this study. The present study further tested the knock-in efficiency at RAB11A locus in K562 cells for larger (4 Kb and 8 Kb) payload sizes using cssDNA. Although lower than 2 Kb payload, both the 4 Kb and 8 Kb payload cssDNAs produced significant knock-in efficiency in a dose-dependent manner (FIG. 2F). Therefore, GATALYST™ gene writing platform with cssDNA outperformed other nucleic acids templates for gene knock-in, and enabled efficient extra-large transgene knock-in.

### Example 5: Highly Efficient iPSC Engineering with cssDNA Donor Template

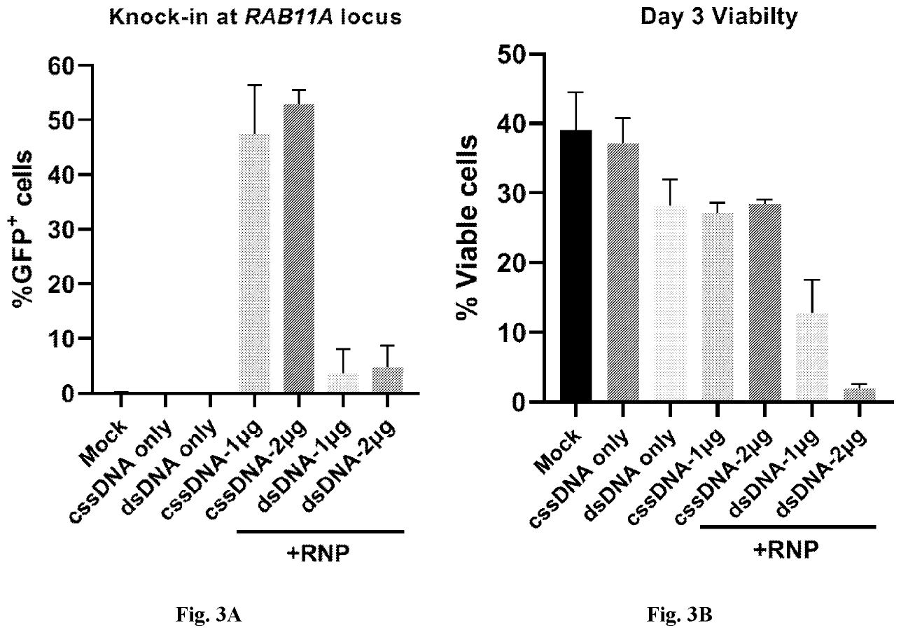

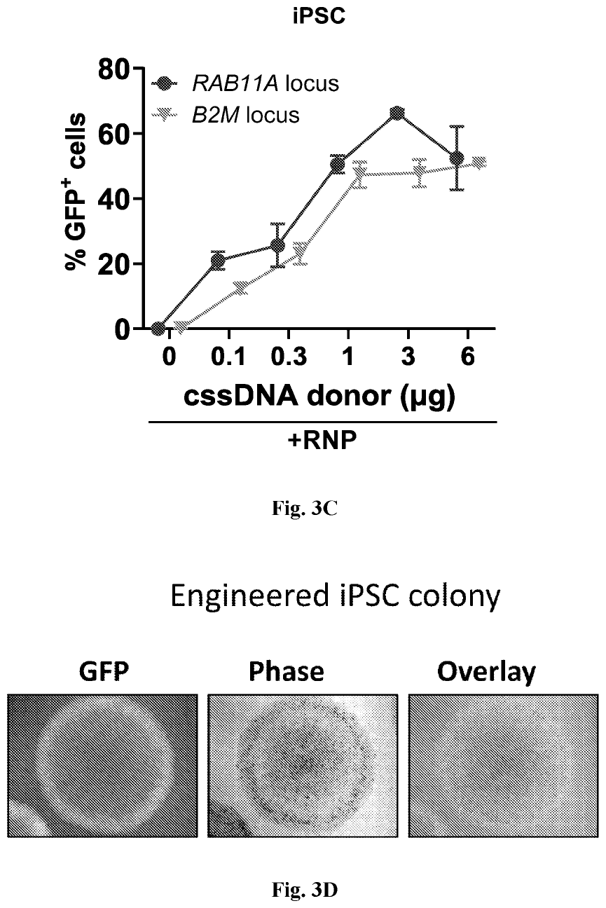

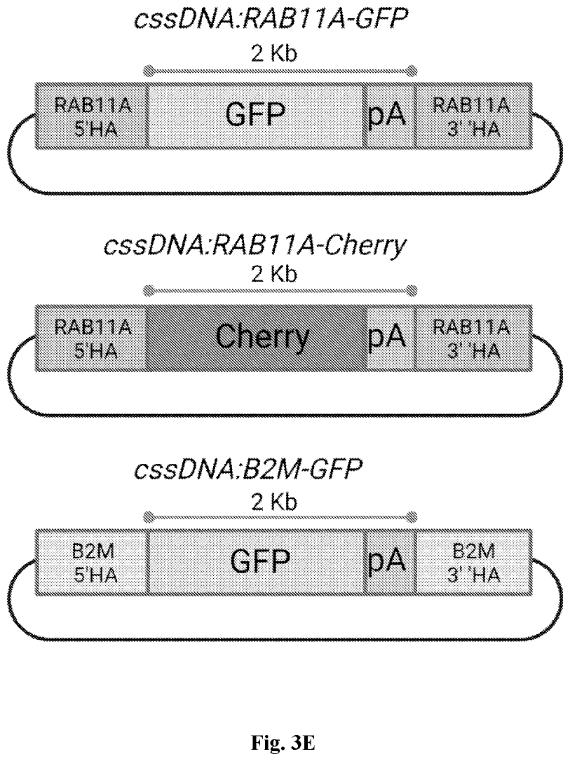

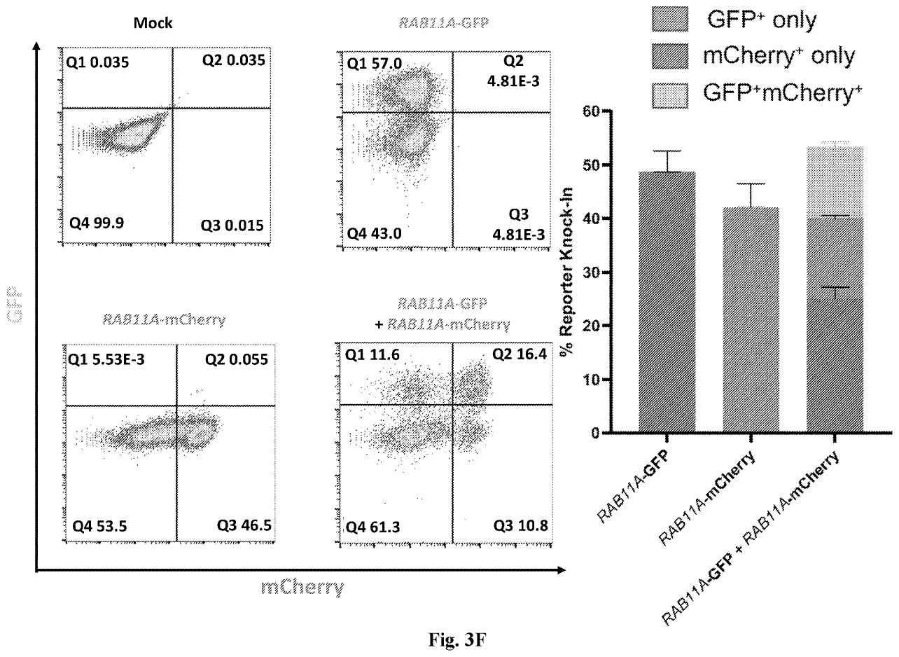

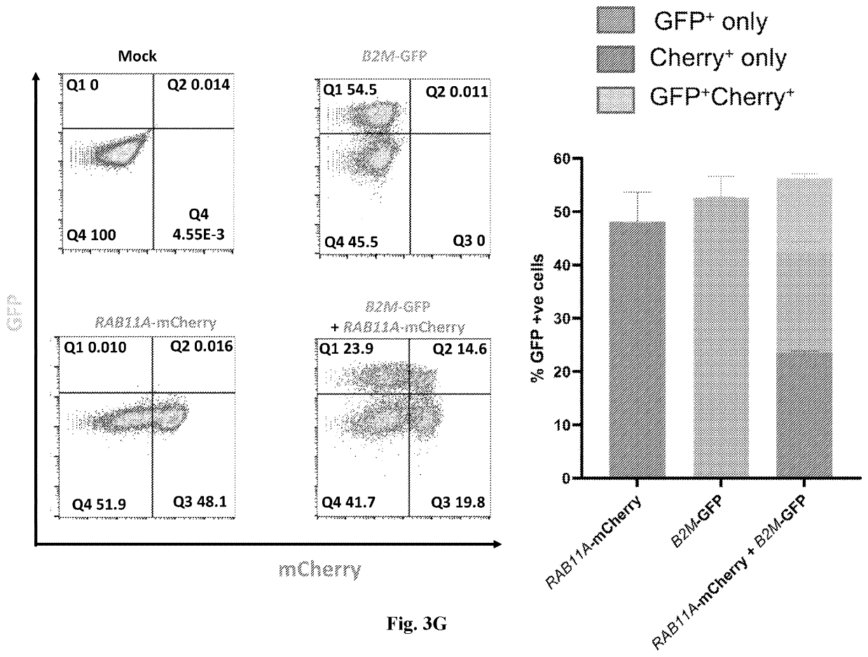

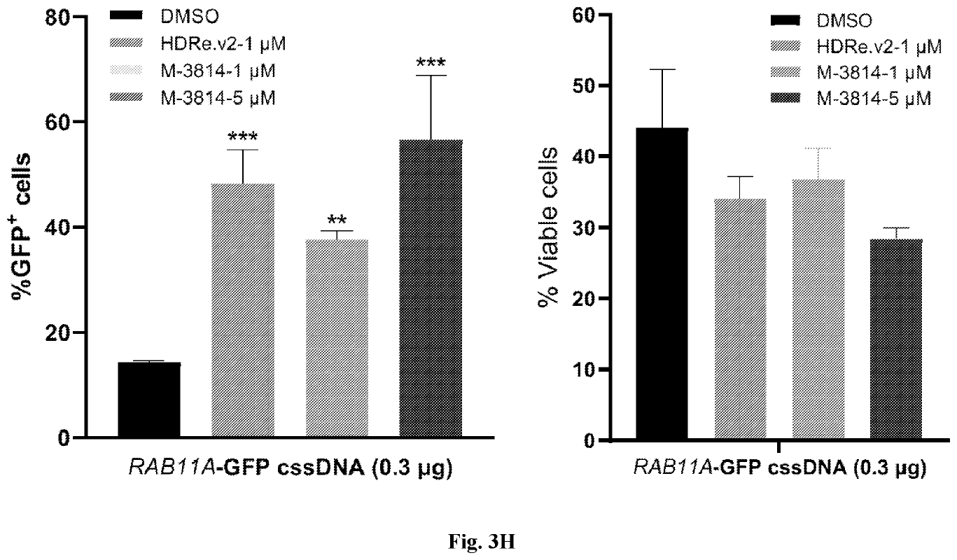

Genetic engineering, especially precise transgene knock-in of induced pluripotent stem cells (iPSCs), holds great promise for gene and cell therapy as well as drug discovery. The present study next sought to test the knock-in with cssDNA donor template. The present study again compared the knock-in efficiency and cell health of cssDNAs vs dsDNAs as an HDR donor templates. In agreement with the previous observations in K562 cells, RAB11A locus targeting GFP cssDNA donor templates outperformed dsDNA donor templates with ˜50% knock-in efficiency and no comprise to iPS cell health (FIGS. 3A-3B). Additionally, knock-in efficiency was tested with a range of cssDNA dosages. The highest knock-in efficiency (over 60%) was observed with 3 μg of RAB11A targeting cssDNA. Similar data was observed for B2M targeting cssDNA mediated knock-in, suggesting the versatile application of cssDNA for genome knock-in at different genomic loci (FIG. 3C). Throughout the process, the engineered iPSC colonies maintained the morphological features characteristic of undifferentiated cells (FIG. 3D). Four cssDNA-engineered iPSC colonies (two with GFP knock-in at RAB11A locus and two with GFP knock-in at B2M locus), when subjected to KaryoStat+assay, showed no chromosomal aberrations when compared against the reference, thus indicating no aneuploidies, submicroscopic aberrations, or mosaic events in cssDNA-engineered iPSC colonies. Furthermore, in a TaqMan® hPSC Scorecard™ Pane assay, the same four engineered iPSC colonies had confirmed expression for the nine panel genes associated with self-renewal, while lacking the expression of f ectodermal, mesodermal, and endodermal genes. Such data demonstrates that cssDNA engineered iPSC colonies maintain their pluripotency.

Bi-allelic genome knock-in at the same locus was previously considered rare, especially for large (multi-kilobase) payload knock-in. To determine the efficiency of bi-allelic integration, the present study engineered two donor cssDNA templates with different fluorescence markers (GFP and mCherry) targeting the same RAB11A locus site in iPSCs (FIG. 3E). As expected, when only one reporter cssDNA was delivered with RNP targeting RAB11A locus, 40-50% reporter knock-in were observed. When two reporter cssDNAs were co-delivered with RAB11A RNP in iPSC, ˜25% of cells were GFP only positive and ˜15% of cells were positive for only mCherry, while ˜15% of cells expressed both fluorescence reporters. Expressing both fluorescence reporters indicated the bi-allelic integration in the cell. Because the single fluorescence positive cells were expected to contain both mono-allelic and bi-allelic integration populations, higher percentage (˜50%) of bi-allelic integration at RAB11A locus is expected in the successfully engineered cell population. In addition, two fluorescence reporter cssDNAs targeting RAB11A and B2M locus, respectively (FIG. 3E) were designed to determine if simultaneous multiplex knock-in at two different loci can be achieved in iPSC. When both cssDNA donor templates were co-electroporated with two different RNP targeting RAB11A and B2M loci, ˜15% of cells expressed both fluorescence reporters (FIG. 3G). This data demonstrates that cssDNAs are superior HDR donor templates to elicit efficient bi-allelic integration at the same locus and multiplex knock-in at different loci simultaneously.

The present study next evaluated small-molecule inhibitors that have been reported to enhance knock-in efficiency, including the DNA-dependent protein kinase (DNA-PK) inhibitor M-3814 and “Alt-R HDR enhancer 2” (HDRe.v2), which is described as a nonhomologous end joining (NHEJ) inhibitor (Kath et al., Mol Ther Methods Clin Dev. 2022 April 12:25:311-330; Shy et al., Nat Biotechnol. 2023 April; 41 (4): 521-531; and Tatiossian et al., Mol Ther. 2021 Mar. 3; 29 (3): 1057-1069). Cells treated for 1 hour after electroporation with either M-3814 or HDRe.v2 (FIG. 3H) demonstrated increased knock-in efficiency in iPSC cells of up to 200-300% compared to untreated controls. Live cell counts were generally unaffected at the chosen concentrations, except for higher doses of M-3814, which demonstrated a roughly 38% reduction in cell viability at day 4 post-electroporation.

### Example 6: Efficient Primary B Cell and CD34⁺ HSPC Engineering with cssDNA

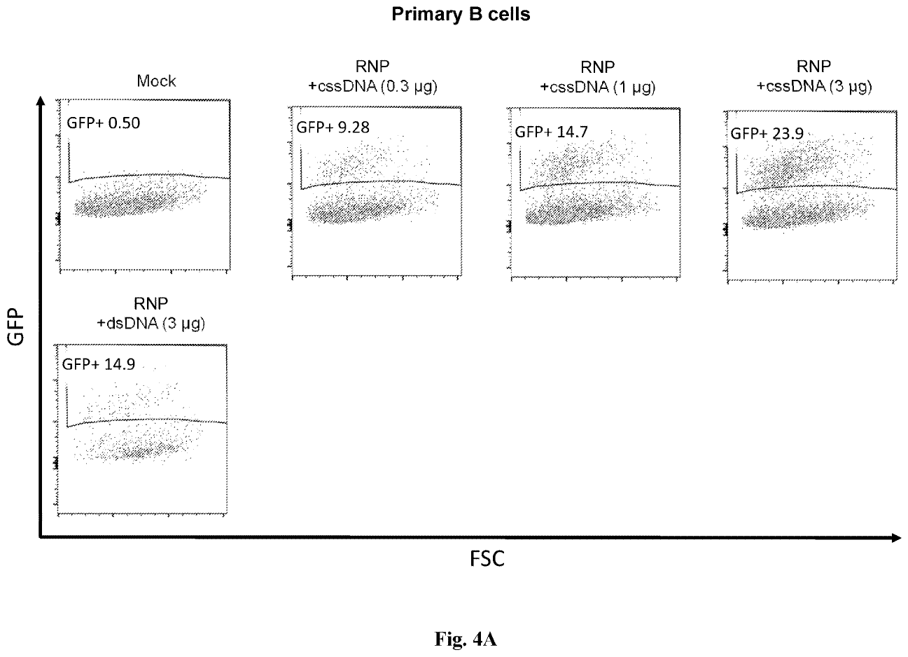

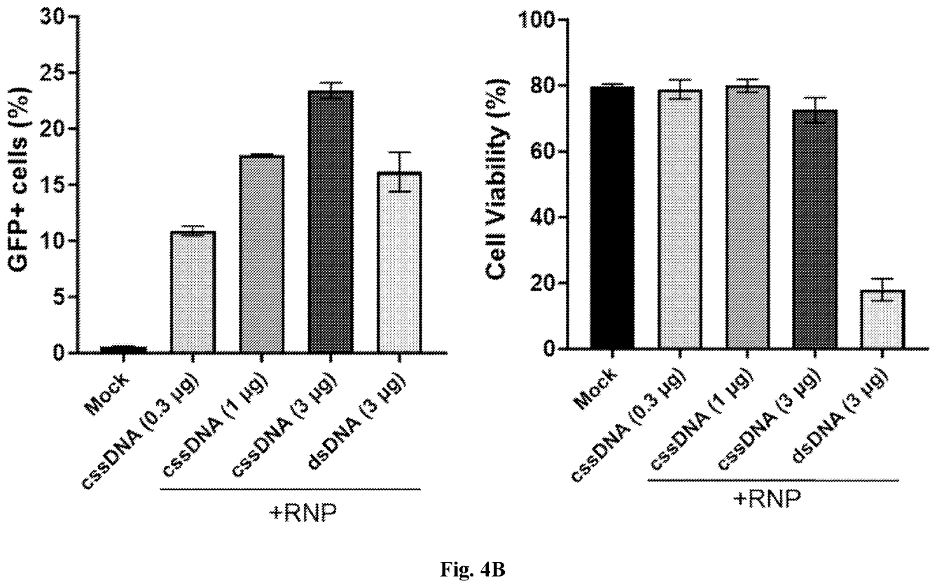

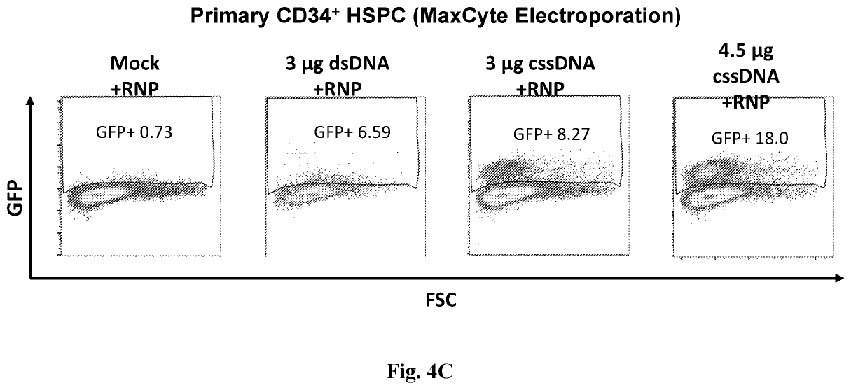

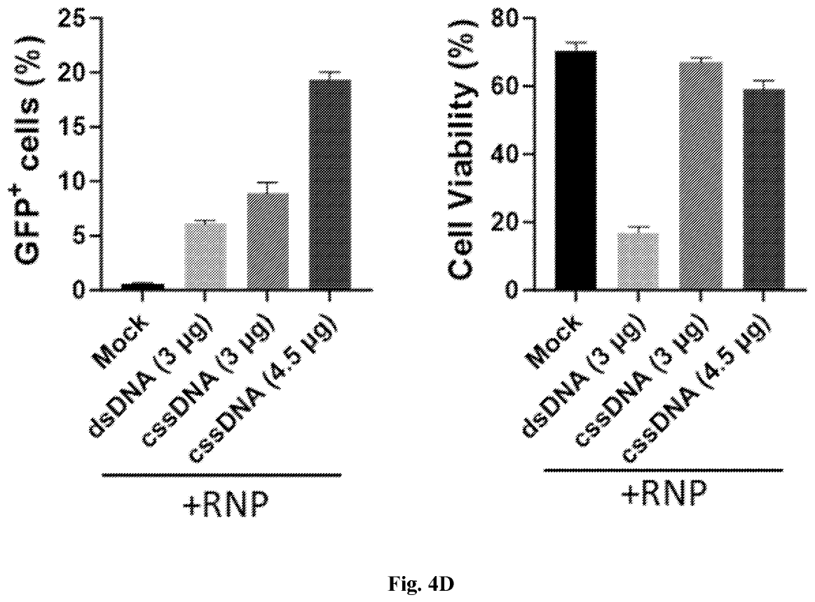

B cells offer attractive opportunities in gene therapy due to their ability to produce high levels of secreted proteins and persist as long-lived plasma cells. B cell precision engineering with CRISPR/Cas9 has shown promising efficiency using HDR donor templates delivered by AAV6. The present study attempted to engineer primary B cells using non-viral cssDNAs as HDR donor templates. GFP reporter-tagged dsDNAs or cssDNAs targeting RAB11A locus used in previous study were co-delivered with RNP into primary B cells. Knock-in efficiency was determined by flow cytometry for GFP⁺ cells. As shown in FIG. 4A and FIG. 4B, B cell engineering with cssDNA HDR donor templates resulted in dose-dependent knock-in, and up to 24% of GFP⁺ cells were achieved with 3 μg of cssDNAs without impacting cell health. However, 3 μg of dsDNA donor templates only resulted in ˜15% of GFP⁺ cells, but cell viability decreased by 80%.

Ex vivo gene therapy based on CD34⁺ hematopoietic stem/progenitor cell (HSPCs) involving lentiviral vectors and NHEJ-based gene-editing has exhibited substantial clinical progress in recent years. However, studies involving HDR-mediated HSPC engineering, especially with non-viral HDR donor template delivery, have not yet made significant advancements. GFP reporter dsDNA and cssDNA donor templates targeting RAB11A locus used in earlier experiments were co-delivered with RNP into primary CD34+HSPC using MaxCyte ATx system. Knock-in efficiency was determined by flow cytometry for GFP⁺ cells. As shown in FIGS. 4C-4D, up to ˜20% knock-in efficiency was observed with 4.5 μg of cssDNAs, and ˜8% of GFP⁺ cells were observed with 3 μg of cssDNAs. HSPC cell viability was generally well maintained for both dosage of cssDNAs. In contrast, 3 μg dsDNA donor templates only resulted in ˜6% of GFP knock-in, while the cell health was largely compromised.

### Example 7: CssDNA Efficiently Engineer Primary T Cells without Inducing Innate Cellular Immune Response

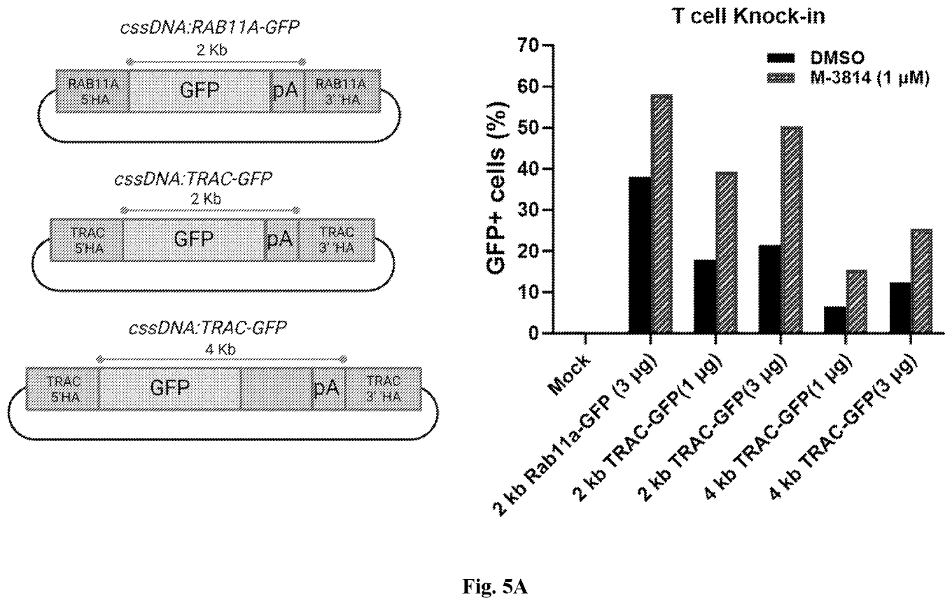

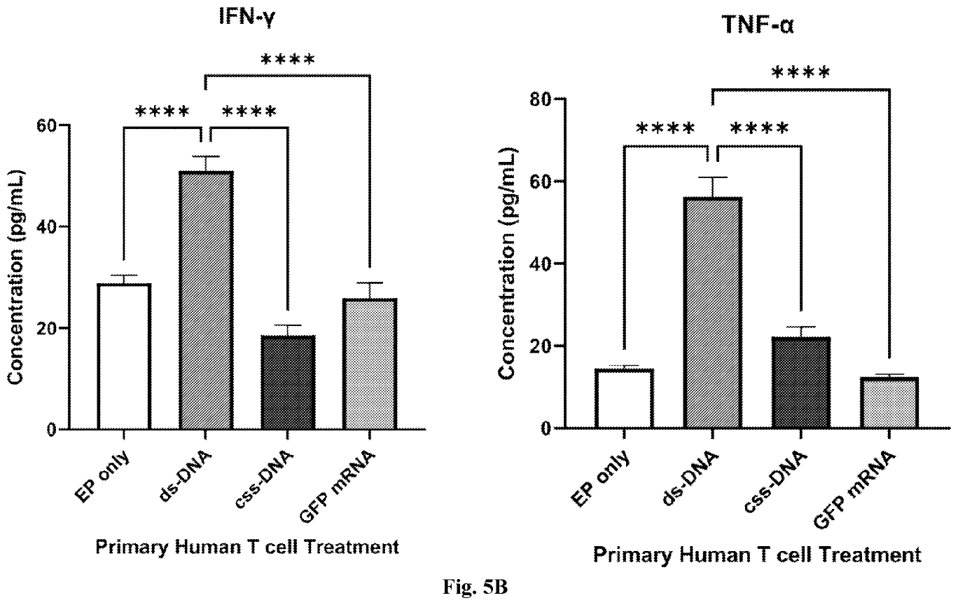

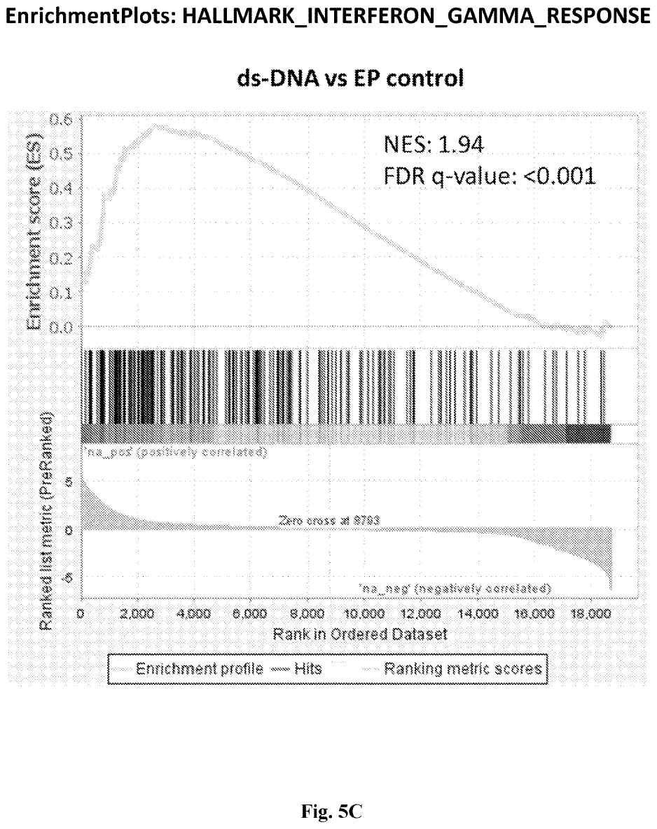

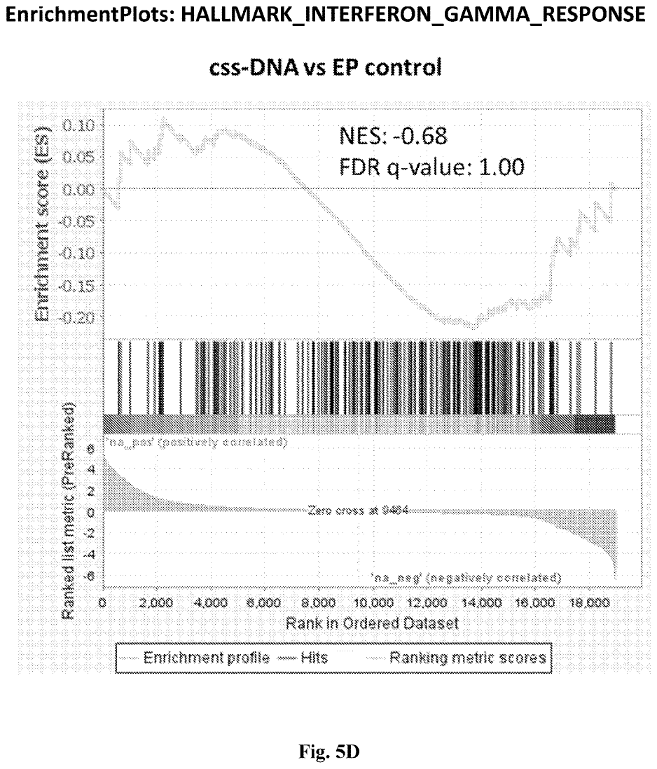

Genetically engineered T cell immunotherapies have provided remarkable clinical success to treat B cell acute lymphoblastic leukemia and have the potential to provide therapeutic benefit for numerous other cancers, infectious diseases, and autoimmunity. Adoptive T cell therapies rely on exogenous gene transfer in primary T cells, resulting in transient or stable transgene expression. The five chimeric antigen receptor (CAR) T cell therapies that have been approved by FDA (Kymriah, Yescarta, Tecartus, Breyanzi, and Abecma) all utilized lentiviral vectors to deliver transgenes in T cells. A variety of non-viral delivery approaches have also been investigated in different stages of preclinical studies. GFP reporter cssDNA HDR donor templates targeting RAB11A locus (RAB11A-GFP), or TRAC locus (TRAC-GFP) were used to engineer primary T cells. The 2 Kb (excluding the homology arms) RAB11A-GFP and 2 Kb or 4 Kb TRAC-GFP cssDNA templates were tested in T cell experiments (FIG. 5A). Primary T cells were co-electroplated with respective RNPs, and knock-in efficiency was determined on Day 7 post electroporation by GFP⁺ cell percentage using flow cytometry. Higher integration efficiency was observed for 2 Kb payload size compared to 4 Kb payload. Consistent with findings in iPSC, DNA-PK inhibitor M-3814 treatment significantly increased knock-in efficiency by 60-150% (FIG. 5A).

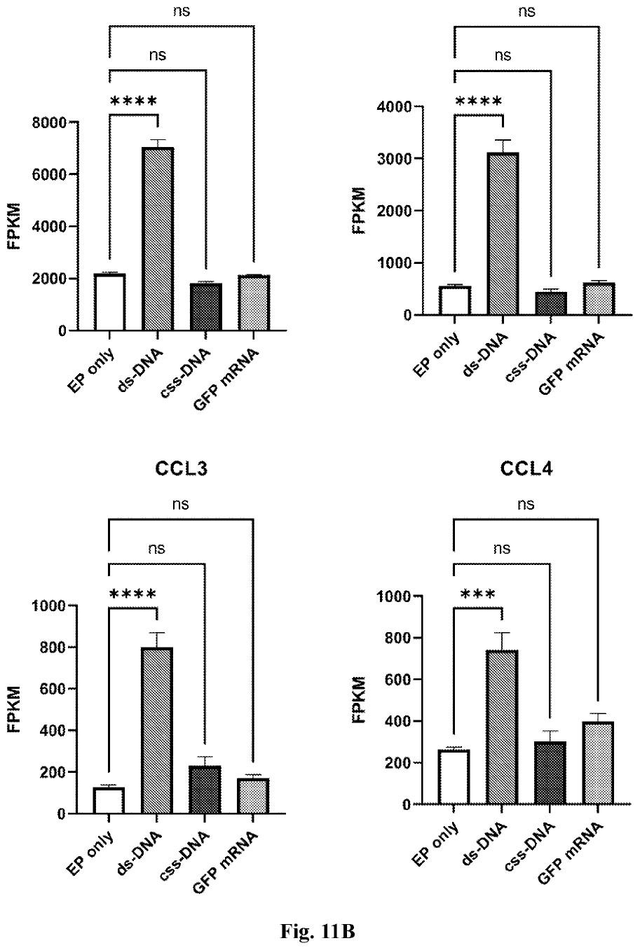

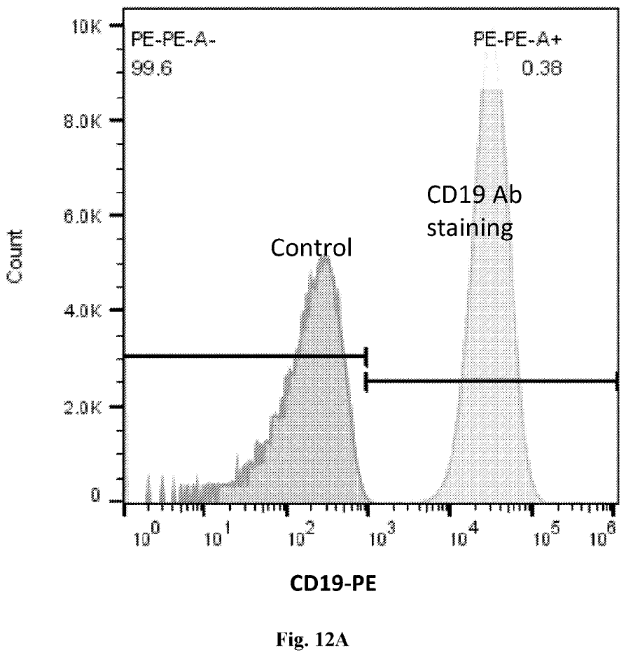

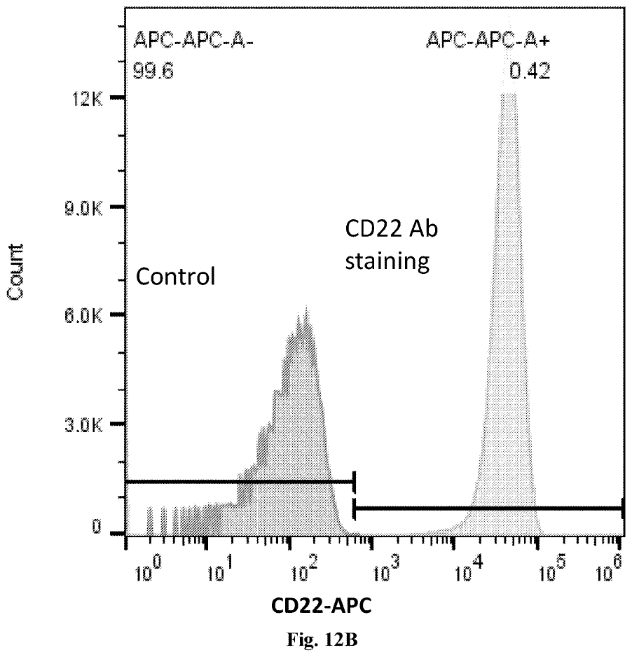

Traditionally, dsDNAs were used as HDR donor templates for precision genome engineering. However, the usage of dsDNA in gene and cell therapy is largely limited by its cytotoxicity, mainly due to triggering cellular innate immune responses mediated by cytosolic DNA sensing pathways, such as Toll-like receptor 9 (TLR9), cyclic GMP-AMP synthase (cGAS), stimulator of interferon genes (STING), etc. The present study compared dsDNA- and cssDNA-mediated proinflammatory response in cultured primary T cells. T cells were electroporated with same amount (1 μg) of dsDNA or cssDNA, GFP mRNA or vehicle (H₂O). 24 hours post treatment, culture medium was collected for cytokine analysis and cell pellets were collected for transcriptome analysis by RNA sequencing. As shown in FIG. 5B, interferon-gamma (IFN-γ) and TNF-γ in culture medium were both significantly increased when T cells were treated with dsDNA, but not by cssDNA or mRNA (FIG. 5B). Consistently, enrichment analysis on the differential expression genes in RNA sequencing analysis demonstrated that dsDNA highly activated intracellular IFN-γ response genes. However, there were no significant changes in IFN-γ response genes when T cells were treated with cssDNA (FIGS. 5C-5D). A panel of innate cellular immune response related genes (OSA1, OSA2, OSA3, OASL, IFIT1, TNF, CCL3, CCL4, etc.) were elevated by dsDNA treatment, but not cssDNA or mRNA (FIGS. 11A-11B). This data demonstrates that unlike dsDNA, cssDNA does not trigger significant cellular proinflammatory response, suggesting it is a much safer HDR donor template.

### Example 8: CssDNA Donor Template are Compatible with Other Nuclease Editors

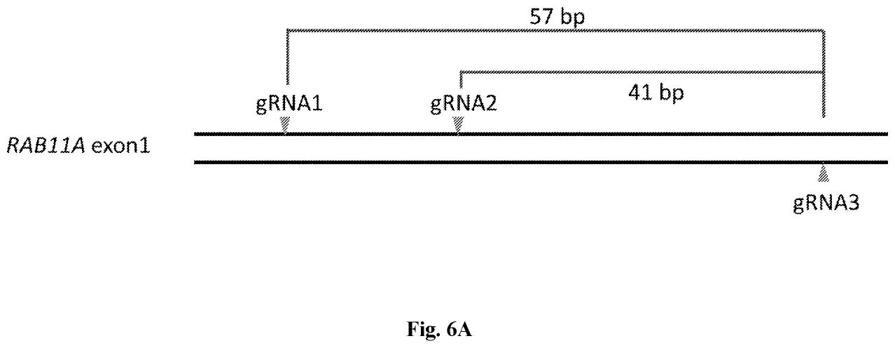

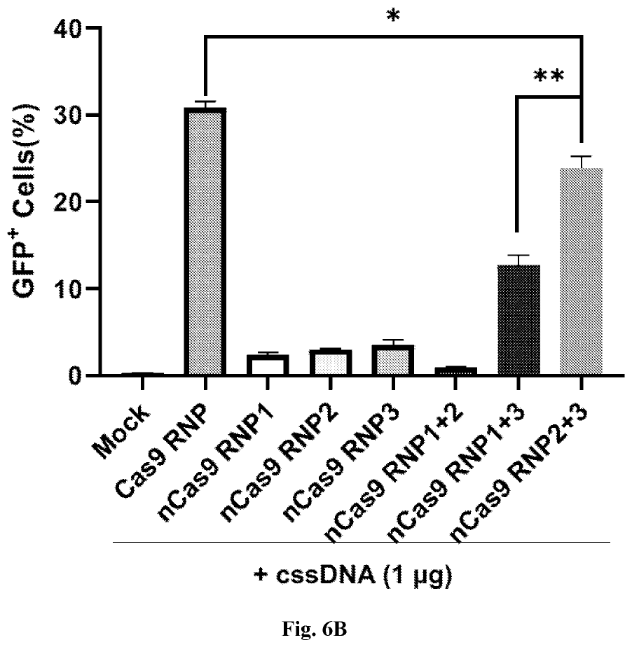

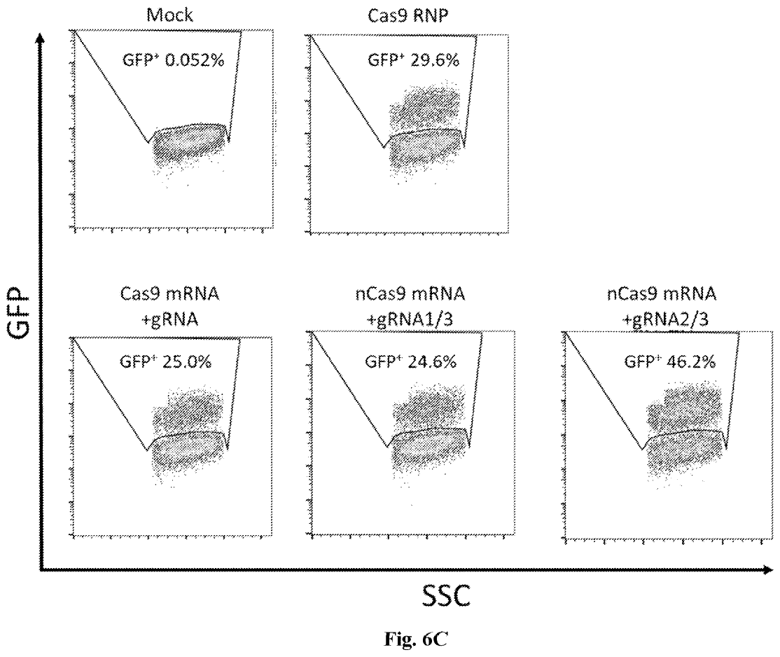

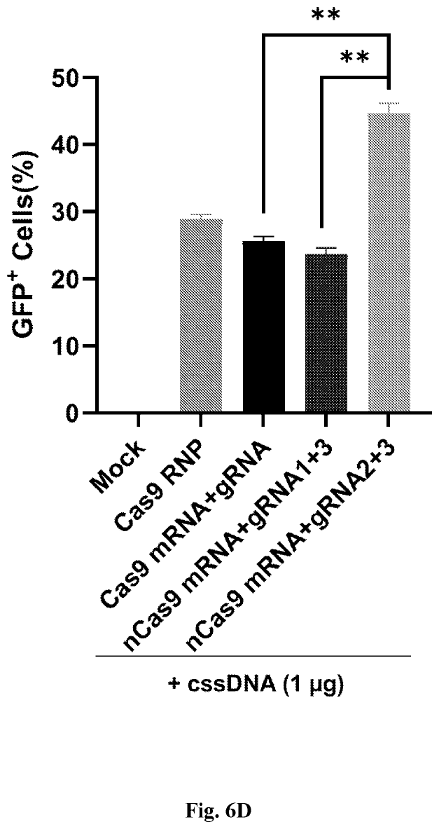

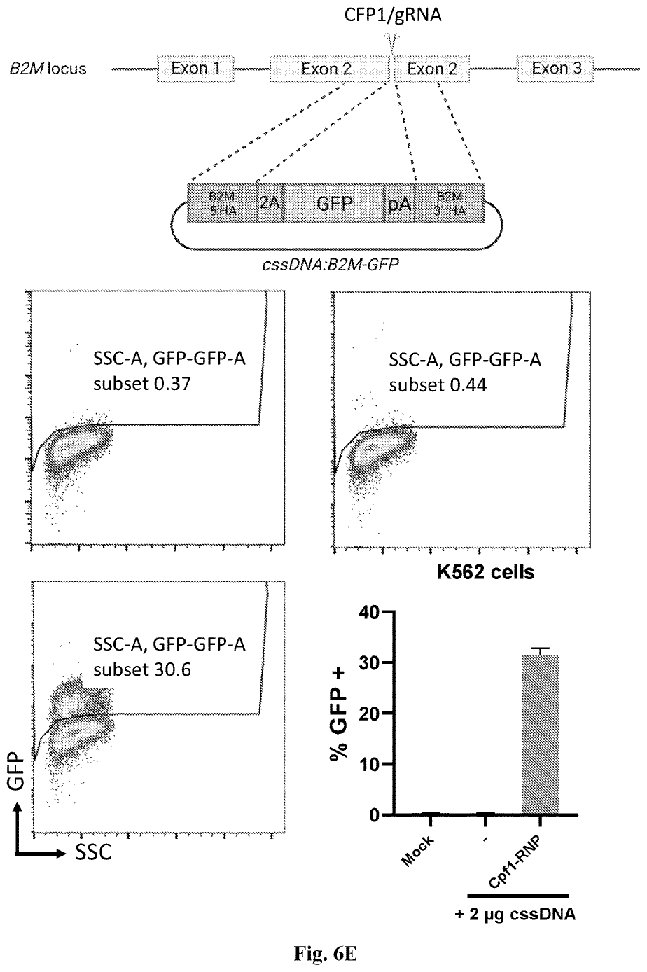

Although Streptococcus pyogenes Cas9 (SpCas9) is the most widely CRSIPR variant used in genome engineering experiments, its application does have certain limitation, such as less stringent protospacer adjacent motif (PAM) sequence which could results in non-specific targeting. The off-target effects could cause detrimental effects in the cells and are especially concerning for clinical applications. Alternative engineered Cas9 variants such as Cas9 nickases have been developed to mutate one of the catalytic domains, thus only nicking a single DNA strand at the desired target, instead of creating a DSB. Paired nickases targeting opposite complementary strands of DNA, each with a different guide RNA, could be used to create a DSB with high fidelity. The use of paired nickases is demonstrated to drastically reduce off-target effects, while maintaining (or sometimes with higher) Cas9 nuclease efficiency. To evaluate the efficiency of cssDNA-mediated genome knock-in for different variants of Cas9 nucleases, double Cas9 nicking by a pair of sgRNAs was used to introduce targeted DSB, which has enhanced genome editing specificity. Following the strategy of Schubert et al., (Sci Rep. 2021 Sep. 30; 11 (1): 19482), two PAM-out configurations with 41-nt and 57-nt nick distances were designed by D10A SpCas9 nickase (nCas9) (FIG. 6A) at exon 1 of RAB11A locus. As expected, GFP cssDNA HDR template targeting RAB11A locus did not show significant GFP knock-in when nCas9 RNP with gRNA1, gRNA2 or gRNA alone (FIG. 6B). Only the two PAM-out configurations with double nCas9 nicking sgRNA1+sgRNA (RNP1+3) or sgRNA2+sgRNA3 (RNP2+3) showed significant GFP knock-in when co-delivered with GFP reporter cssDNA HDR templates in K562 cells (FIG. 6B). RNP2+3 pair showed significantly higher efficiency than RNP1+3, although lower efficiency compared to wild type spCas9 (wtCas9) with RNP, which induced blunt DSB. As a control, RNP1+2 which both nicked the same targeting strand did not induce any knock-in. This data demonstrates that cssDNA donor templates are compatible with different Cas9 variants to provide a versatile toolbox for efficient and safe genome knock-in.

The present study further tested the efficiency when delivering Cas9 by mRNA rather than protein (RNP). Similar knock-in efficiency was observed with Cas9 RNP and Cas9 mRNA (FIGS. 6C-6D). When the gRNA1+3 and gRNA2+3 was co-delivered with nCas9 mRNA, the knock-in efficiency was significantly higher than when nCas9 was delivered by protein (RNP) (FIGS. 6B and 6D). nCas9 mRNA gRNA2+3 was more effective than Cas9 mRNA gRNA for cssDNA-mediated knock-in. These results indicate that GATALYST gene writing using cssDNA HDR donor template is agonistic to various nucleases and can be delivered in different forms (protein and mRNA). In certain embodiments, mRNA delivery can result in more durable Cas9 expression, and is expected to have a higher nuclease activity to induce two-nicking DSBs compared to protein delivery. The present data aligns with this expectation, as cssDNA HDR donor-mediated knock-in is significantly higher than RNP delivery, further supporting cssDNA as a superior editing tool.

CRISPR from Prevotella and Francisella 1 (Cpf1) endonuclease, previously known as Cas12a, has recently gained more popularity as an advanced substitute for CRISPR/Cas9 and more efficient genome-editing tool (Moon et al., Nat Commun. 2018 Sep. 7; 9 (1): 3651) due to its smaller size compared to Cas9 and requirement of shorter CRISPR RNA (crRNA). Guided by a single RNA, Cpf1 nuclease cuts the DNA at the proximal end of the PAM (5′-TTTN-3′), by introducing 5 base pair (bp) staggered cuts. The present study screened multiple crRNA for Cpf1 at human B2M locus and identified one Cpf1-crRNA with over 95% cleavage efficiency. A GFP reporter cssDNA construct was designed and synthesized as donor template for HDR knock-in in K562 cells. As shown in FIG. 6E, up to 30% GFP knock-in at B2M locus was achieved with 2 μg of cssDNA donor template when co-delivered with Cpf1/crRNA RNP. These demonstrated that cssDNAs can serve as universal donor templates to be used in multiple nuclease editor systems.

### Example 9: Efficient CAR-T Engineering with Durable Cancer Cell Lysis Function Using cssDNA Donor Template

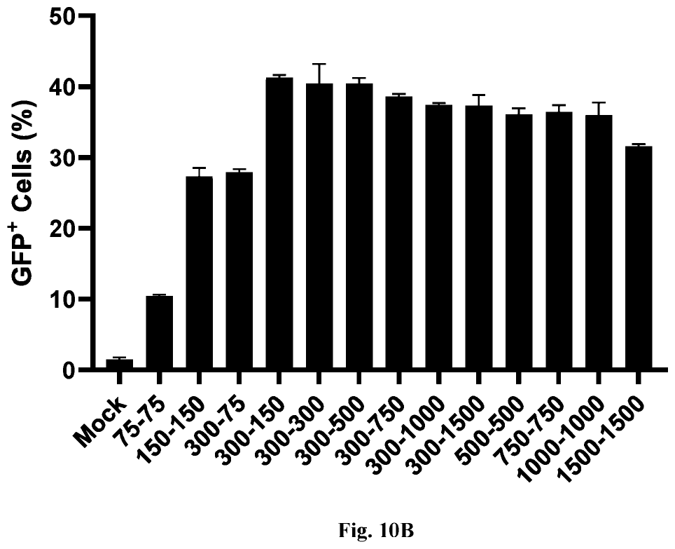

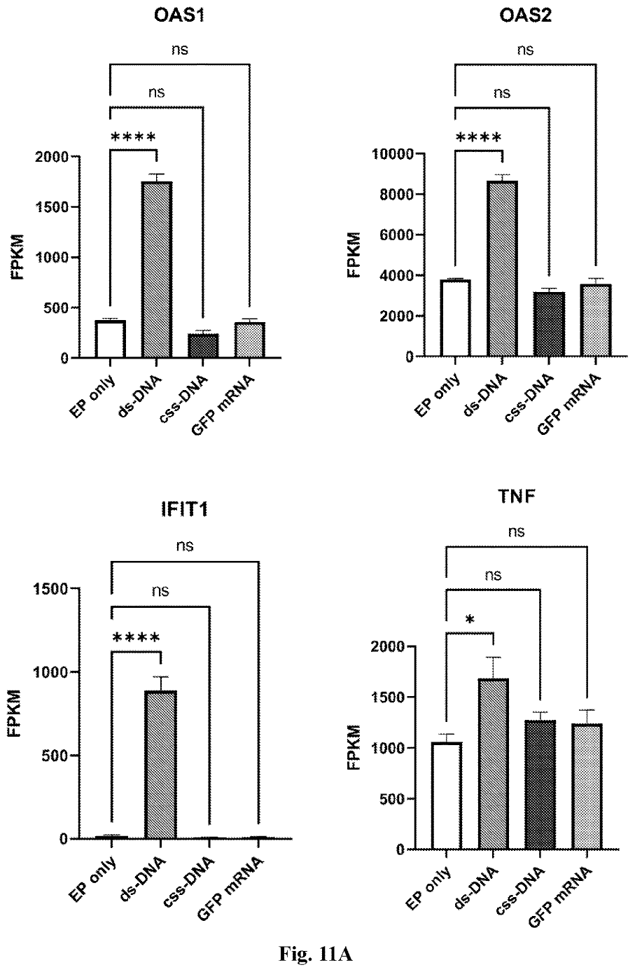

Finally, the present study sought to engineer T cells with chimeric antigen receptor (CAR) using non-viral cssDNA donor templates to demonstrate the therapeutic potential of cssDNAs (FIG. 7A). The present study engineered bi-specific CARs targeting CD19 and CD22 (Qin et al., Mol Ther Oncolytics. 2018 Nov. 6:11:127-137 and Spiegel et al., Nat Med. 2021 August; 27 (8): 1419-1431), which offered great potential to treat CD19 CAR resistance relapsed or chemotherapy-refractory (relapsed/refractory) B cell malignancy resistance, into the endogenous TRAC locus. Targeting a CAR to the TRAC locus greatly enhanced the potency of CAR-T cells in preclinical studies and delayed effector T-cell differentiation and exhaustion. Anti-CD19×CD22-CAR cssDNA targeting TRAC locus was co-electroporated with spCas9 protein and TRAC sgRNA. Day 7 post electroporation, CAR expression was determined by Protein L binding. Approximately 33% knock-in efficiency (percentage of CAR+ cells) was achieved with 2 μg of cssDNA donor templates. When treated with DNA-dependent protein kinase inhibitor M-3814, knock-in efficiency further increased to 56.3% (FIG. 7B). Similar data was collected with CD19 CAR specific detection reagent for CAR detection in flow cytometry. Protein L was used for all the following CAR detection experiments. To benchmark against the engineering with donor template delivered by rAAV in previous studies, equivalent TRAC locus cssDNA and AAV6 vectors encoding an anti-CD19×CD22-CAR were made, and direct comparisons demonstrated efficient knock-in with both approaches. Although, consistently higher CAR expression efficiency was observed with AAV6 vector templates (75-80%) than with cssDNA templates (40-50%) (FIG. 7C). Both cssDNA and AAV6 engineered T cells expanded 25-30-fold during 7 days of culture, only slightly lower than un-engineered mock T cells (FIG. 7D). On Day 7 post engineering, the cytotoxicity of engineered CAR-T cells was examined. In vitro assays demonstrated efficient CAR-T cell killing of CD19+ and CD22+NALM6 cells (FIGS. 10A-10B), in contrast to the mock engineered T cells from the same donor (FIG. 7E). Interestingly, although at lower knock-in efficiency, the target cell killing activity of cssDNA engineered CAR-T were significantly higher than AAV6 engineered CAR-T cells at 24-hour time point, suggesting higher potency of cssDNA CAR-T cells than AAV6 CAR-T cells. Furthermore, when in vitro cell killing effects were monitored for extended time (96 hours), target cells re-grew significantly with un-engineered T cells as well as with AAV6 engineered CAR-T cells at most conditions except 6:1 effector: target ratio in the killing assay (FIGS. 7F-7G). However, target cell re-growth was only observed in the lowest effector: target ratio with cssDNA CAR-T cells killing assay. The data demonstrates that despite lower CAR knock-in efficiency, cssDNA engineered CAR-T cells present with higher potency and higher durability against CD19+CD22+target B cell leukemia cells. cssDNA engineered CAR-T cells present with higher potency and higher durability against CD19⁺CD22⁺ target B cell leukemia cells. To evaluate the in vivo function of the cssDNA engineered CD19/CD22 dual CAR-T, the present study transplanted 3×10⁶ CAR⁺ T cells vial tail vein delivery in the leukemia NALM-6 mouse xenograft model (FIG. 7H). Compared with non-engineered T cells, cssDNA engineered non-viral CAR-T significantly decreased tumor burden of NALM6-bearing mice as indicated by reduced luciferase expression from engrafted NALM6+tumor cells, as well as improved survival for all the tumor bearing mice transplanted with CD19/CD22 dual CAR-T cells (FIGS. 7H-7I).

### Example 10

NK cells differ from T cells in that they do not rely on a matching human leukocyte antigen to function, making allogeneic transfer safe from graft-versus-host diseases. Genetic engineering of primary NK cells to increase killing activity and reduce tumor evasion is an attractive immunotherapy direction. However, genetic engineering of primary NK cell is challenging with conventional methods because NK cells are highly sensitive to exogeneous DNAs that were used to deliver genes of interest. Retroviral transduction was proved as an effective method, but it requires high viral titer and poses safety concerns. A Cas9 nucleofection protocol co-electroporation with PCR-amplified double-stranded DNA donor template containing GFP reporter gene was recently reported, which resulted in 4-10% GFP reporter knock-in at various genomic loci (i.e., ACTB, RAB11A or CD96). The present study sought to determine the NK cell engineering with cssDNA. First, the RAB11A-GFP cssDNA donor template targeting RAB11A locus was used to demonstrate the effectiveness of NK cell engineering. Primary NK cells isolated from the healthy donor were expanded in the presence of mitomycin C irradiated Epstein-Barr virus-transformed lymphoblastoid cell line (EBV-LCL) feeder cells (FIG. 8A). Freshly isolated NK cells or recovered from cryopreservation expanded up to 10,000-fold over 25-40 days when co-cultured with feeder cells (FIG. 8B). NK cells were electroporated on Day 10-14 of expansion with RAB11A targeted Cas9 RNP in the presence of RAB11A-GFP cssDNA donor template. The GFP knock-in efficiency was determined by flow cytometry ˜Day 7 post electroporation. The percentages of GFP positive cells were 34.3% and 36.9% when 2 μg and 4 μg of donor templates were used, respectively (FIG. 8C). The NK cell viability was not compromised across different groups (FIG. 8D). The present study then constructed a cssDNA donor templates to knock-in a CD19.CAR 1928z-1xx at the RAB11A locus. The CD19.CAR 1928z-1xx has superior anti CD19+target tumor cell activity. As shown in FIG. 8D, NK cell viability recovered quickly, reaching 80-90% 6-7 days post engineering. NK cell expansion was similar across treatment group. Flow cytometry data on Day 7 post engineering demonstrated over 20% of CD19.CAR knock-in efficiency with 2 μg of cssDNA donor template (FIG. 8E). The cytolytic activity of engineered NK cells against CD19⁺ NALM6 target cells was assessed using Cell Counting Kit-8 mediated in vitro cytotoxicity assay at different ratios of engineered NK cells and target cell (E/T ratios) when co-cultured for 4 hours. The 1928z-1xx engineered primary NK cells have significantly higher target cell killing function compared to mock and RNP groups (FIG. 8E). These data suggest cssDNA is a superior non-viral donor template for primary NK cell engineering.

### Example 11

Single-stranded DNA (ssDNA) exists in nature, which only commonly occurs in viruses or in a transient during dsDNA replication process. Synthetic biology is an emerging field of genome engineering for specific purposes in medicine, manufacturing, and agriculture. In the past several years, ssDNA has attracted attention across the genetic engineering field, mainly due to its low cellular toxicity and high insertion efficiency in CRISPR/Cas-based genomic editing. Although lssDNA can be chemically synthesized or produced in vitro by enzymatic processes, these methods can be costly, lack scalability, and currently cannot create long molecules (over 5 Kb). In addition, the produced linear strands are prone to exonuclease degradation. The manufacturing of high purity cssDNAs up to 20 Kb in length from engineered phagemids provides a low-cost alternative. cssDNAs are more resistant to exonuclease activity, which may prove important for therapeutic applications.

Thus, the new technological developments in synthetic cssDNA production can be made bio-orthogonal and scalable, enabling future applications to novel therapeutics. The present cssDNA purification process from engineered phagemid is research grade at small scale and relatively low yield. However, its production was proven to be amendable to scalable bath fermenter.

Accumulating evidence suggested that genome integration with the existing CRISPR/Cas9-mediated HDR technology in engineered cells is often mono-allelic. However, for applications in monogenic disease mutation correction, gene therapy, transgenic model animal generation, and agriculture manufacturing, it is highly desirable for biallelic genome modification. The instant cssDNA is a superior HDR donor template, not only for its low toxicity and high knick-in efficiency, but also for its observed high level of bi-allelic integration, allowing its application for many versatile applications in basic research, gene and cell therapy, agriculture, and climate change.

Overall, phagemid-derived cssDNAs were demonstrated to be superior HDR donor templates offering highly efficient genome knock-in with low cellular toxicity. cssDNA production is bio-orthogonal and scalable, with the capability to generate cssDNA lengths well beyond the packaging capacity of current viral vectors. cssDNAs were shown to be well suited for multiple nucleases, in multiple cell types at multiple genetic loci. In immune cell therapy, ˜50% targeted CAR knock in on TRAC locus in primary T cells has been proved using cssDNAs as HDR donor templates. cssDNA engineered CAR-T cells showed higher potency and higher durability to target tumor cells, when compared to CAR-T cells engineered with donor templates delivered by AAV6. These advantages make cssDNAs attractive HDR donor templates for efficient insertion of large DNA payloads in a variety of disease-relevant cell types and can be leveraged for basic research and gene and cell therapies.

### Example 12: Materials and Methods

Generation of HDR Template Circular Single-Stranded DNA from M13 Phage

Donor template sequences (transgene sequence flanked with 5′ and 3′ homology arms at 300-500 nt in length) are constructed as dsDNA and placed into phagemid vector by Golden Gate Assembly. An XL1-Blue E. coli Strain was co-transformed with the M13 helper plasmid and phagemid containing double-stranded donor template and selected in agar plate with kanamycin (50 μg/mL), carbenicillin (100 μg/mL). A single colony was selected and grown for ˜24 hours (37° C., 225 rpm) in 250 mL 2×YT media (1.6% tryptone, 1% yeast extract, 0.25% NaCl) to reach OD600 between 2.5-3.0. The bacteria were pelleted by centrifugation and the phage particles in the supernatant were precipitated by PEG-8000. The precipitated phage particles were then pelleted by centrifugation, washed, and lysed in 20 mM MOPS., 1M Guanidine-HCl and 2% Triton X-100. The cssDNAs released from phage were then extracted with NucleoBond Xtra Midi EF kit (Macherey-Nagel) following the manufacturer's instructions. The concentration of cssDNAs was determined by Nanodrop for ssDNA and the yields are 10 μg per ml of liquid culture. Ratios of Absorbance (A260 nm/280 nm and 260 nm/230 nm) reflect consistent purity (1.8 and >2, respectively) from serial preps. Recombinant cssDNA is verified by DNA sequencing using custom-designed staggered sequencing primers for complete coverage.

### Cell Culture

K562 (ATCC) and NALM6 (ATCC) cells were maintained in RPMI-1640 media with 10% FBS and 1% penicillin and streptomycin. HEK293T (ATCC) cells were cultured in Dulbecco's modified Eagle's medium supplemented with 10% fetal bovine serum (FBS) and 1% penicillin and streptomycin (Gibco). iPSCs (Thermo Fisher) were cultured in complete StemFlex (Thermo Fisher) media in vitronectin-coated flask. iPSC colonies were checked regularly and passaged using ReLeSR (StemCell Technologies) every 3-4 days of culture. iPSCs were ready for electroporation after 2-3 passages. Human primary CD34+HSPCs were isolated from fresh Leukopak using CD34 MicroBead Kit in MultiMCAS Cell24 Separator Plus (Miltenyi). The cryopreserved HSPCs were thawed, washed, and cultured in StemSpan SFEM II medium supplemented with StemSpan™ CD34+Expansion Supplement and UM729 (StemCell Technologies). HSPCs were ready for electroporation after 4-5 days in culture. Human primary B cells were isolated using StraightFrom Leukopak CD19 MicroBead Kit in MultiMCAS Cell24 Separator Plus (Miltenyi). Cryopreserved B cells were thawed, washed, and cultured and activated in ImmunoCult™-XF B Cell Base Medium supplemented with ImmunoCult™-ACF Human B Cell Expansion Supplement (StemCell) for 4 days before electroporation. Human primary T cells were isolated using StraightFrom Leukopak CD4/CD8 MB Kit in MultiMCAS Cell24 Separator Plus. T cells were cultured and expanded in TexMACS Medium (Miltenyi) supplemented with 200 IU/mL Human IL-2 IS (Miltenyi). T cells were activated for 2 days with T Cell TransAct (Miltenyi) before electroporation.

All cells were maintained in a humidified incubator at 37° C. and 5% CO₂, unless otherwise specified. Cell count viability was determined using Via2-Cassette in NucleoCounter® NC-202 (ChemoMetec) on specified days after engineering.

### Electroporation

All HEK293T, K562, iPS, B and T cell electroporations were performed using the Amaxa™ 96-well Shuttle™ with the 4D Nucleofector (Lonza). HSPC electroporation weas performed in MaxCyte ATx. 25 picomole of sNLS-SpCas9-sNLS Nuclease (Aldevron) or D10A Cas9 nickase (nCas9) (ThermoFisher) along with 50 picomol of sgRNA (for SpyCas9, synthesized at Integrated DNA Technologies) were used per reaction. Cas9 nucleases and sgRNAs were precomplexed in supplemented Nucleofector® Solution for 20 min at room temperature and the RNP solution was increased to a final volume of 2.5 μL (10×) per electroporation reaction. For mRNA delivery nucleases, 1 μg of Cas9/nCas9mRNA was co-electroplated with 50 picomol of sgRNA. For electroporating K562 cells, an SF Cell Line 4D-Nucleofector™ Kit and 250,000-500,000 cells per reaction were used. For electroporating HEK293T cells, an SF Cell Line 4D-Nucleofector™ Kit and 200,000-300,000 cells per reaction were used. For electroporating iPSC cells, 100,000 cells per reaction were used with a P3 Primary Cell 4D-Nucleofector™ Kit. For electroporating primary B cells, 1,000,000 cells per reaction were used with a P3 Primary Cell 4D-Nucleofector™ Kit. For electroporating primary T cells, 2,000,000 cells per reaction were used with a P3 Primary Cell 4D-Nucleofector™ Kit. For electroporating primary HSPC cells, 1,250,000 cells per reaction were used with a 50 μL reaction cuvette in MaxCyte ATx. The indicated amount of HDR donor template (cssDNA or dsDNA) were used in co-electroporation with RNP or Cas9/nCas9 mRNA and sgRNA. After electroporation, the cells were placed in a humidified 32° C. incubator with 5% CO₂ for 12-24 hours, followed by transferring to 37° C. incubator for 3-10 additional days. For electroporating primary NK cells, ˜500,000 cells per reaction were used with a P3 Primary Cell 4D-Nucleofector™ Kit. The indicated amount of HDR donor template (cssDNA or dsDNA) were used in co-electroporation with RNP or Cas9/nCas9 mRNA and sgRNA. After electroporation, the cells were placed in a humidified 32° C. incubator with 5% CO₂ for 12-24 hours, followed by transferring to 37° C. incubator for 3-10 additional days.

Quantification of dsDNA or cssDNA by qPCR from Electroporated K562 Cells.

Following from 1-10 days after electroporation, K562 cells were pelleted, and total DNA was isolated by PureLink Genomic DNA Mini Kit (Thermo Fisher). 20 ng of DNA was used for quantification of cssDNA or dsDNA using TaqMan Multiplex Master Mix following the manufacturer's recommendations. Amplification was detected in a QuantStudio 12K Flex Real-Time PCR System instrument from ThermoFisher. cssDNA and dsDNA specific Taqman probes purchased from IDT are shown in Table 1 below. The relative remaining DNAs were normalized using 24Ct method.

### Flow Cytometry Analysis

All flow cytometry was performed on an Attune NXT flow cytometer with a 96-well autosampler (ThermoFisher Scientific). Unless otherwise indicated, cells were collected 4-7 days post electroporation, resuspended in fluorescence-activated cell sorting (FACS) buffer (2% FBS in PBS) and stained with 7-AAD (BioLegend), and the indicated cell-surface marker. To obtain comparable live cell counts between conditions, events were recorded from an equivalent fixed volume for all samples. Data analysis was performed using FlowJo_v10.8.0_CL software with exclusion of subcellular debris, singlet gating and live: dead stain. Analyzed graphs were produced with Prism 9 (GraphPad).

### GFP-NALM6 Stable Cell Line Generation

NALM6-GFP stable cell lines were generated by knock-in EF1a-GFP in AAVS1 locus using cssDNAs as donor templates. NALM6 cells were electroporated using Amaxa™ 96-well Shuttle™ in 4D Nucleofector with 25 μmol of Cas9 and 50 μmol of gRNA targeting AAVS1 locus, and 2 μg of cssDNA containing EF1a driven GFP sequence flanked with AAVS1 5′ and 3′ homology arms. Engineered cells were culture for 3-weeks and flow sorted to dispense single GFP-positive cells into individual wells of 96-well plates in BD FACSAria™ III (BD Biosciences). NALM6-GFP stable cells from a single colony were expanded. NALM6-GFP single clone stable cells were used in in vitro cell cytotoxicity assay.

### CAR-T Cell Engineering

48 hours after initiation and activation, T cells were electroporated using Amaxa™ 96-well Shuttle™ in 4D Nucleofector. 2×10⁶ cells were mixed with 25 pmol of Cas9 and 50 pmol of gRNA (RNP) into each well. For cssDNA engineered cells, 2 μg of cssDNA encoding bi-specific CD19×CD22 CAR was electroporated with RNP targeting TRAC locus. For AAV6-medaited engineering, recombinant AAV6 donor vector (manufactured by PackGene) was added to the culture immediately after electroporation, at 2×10⁴ multiplicity of infection. Following electroporation, cells were diluted into culture medium in the presence of T Cell TransAct with DMSO or 1 μM M-3814, and incubated at 32° C., 5% CO₂ for 24 hours. Cells were then washed and subsequently transferred into G-Rex 24 Multi-Well Cell Culture Plate (Wilson Wolf) in standard culture conditions at 37° C., 5% CO₂ in IL-2 (200 IU/mL) supplemented TexMACS medium and replenished every 3-4 days. CAR expression was determined using PE labelled Protein L (ACROBiosystems), or Biotin conjugated CD19 antigen fused recombinant human IgG1 Fc (Miltenyi) in PBS with 2% BSA before flow analysis.

### In Vitro Cell Killing

The cytotoxicity of T cells transduced with a bi-specific CD19×CD22 CAR was determined by IncuCyte Cytotoxicity Assay with Annexin V Red Reagent for apoptosis (Sartorius) using NALM-6 stably expressing GFP served as target cells. On Day 7-9 post electroporation, the CAR-T effector (E) and tumor target (T) cells were co-cultured in triplicates at the indicated E/T ratio using flat bottom poly-L-ornithine (Sigma) coated 96-well plates with 1×10⁴ target cells in a total volume of 200 μL per well in NALM-6 medium. The number of effector CAR T cells was calculated based on the percentage of CAR-positive cells. Target cells alone were plated at the same cell density to determine cell proliferation. Plates were incubated at 37° C. and 5% CO₂ for up to 4 days. Four images were recorded per well every 2 h and analyzed using IncuCyte cell by cell analysis software module (Sartorius). The killing potency of the CAR-T cells was assessed by comparing the percentage of Annexin V positive target cells over time relative to the total number of target cells.

### In Vivo CAR-T Mouse Xenograft Study

In vivo analysis of CAR activity was conducted using a xenograft model with 6- to 8-week-old NOD/SCID/IL-2Ry-null (NSG) female mice (Jackson Laboratory). Mice were inoculated with 0.5×10⁶ Firefly luciferase labelled NALM6 tumor cells intravenously via tail vein on day-3. On day 0, CAR-engineered T cells or mock-transduced T cells were injected intravenously as indicated. Mice received luciferin-D intraperitoneal injections and were imaged using in vivo imaging system (IVIS) technology.

Sequences of gRNA, Primers and Probes

The sequences of gRNA, primers and probes used in the present study are listed below in Tables 1-2:

TABLE 1
sgRNA Sequences

| sgRNA | Sequence |
|---|---|
| RAB11A sgRNA | GGUAGUCGUACUCGUCGUCG (SEQ ID NO: 1) |
| B2M sgRNA | GGCCACGGAGCGAGACAUCU (SEQ ID NO: 2) |
| TRAC sgRNA | AGAGUCUCUCAGCUGGUACA (SEQ ID NO: 3) |
| AAVS1 sgRNA | GGGGCCACUAGGGACAGGAU (SEQ ID NO: 4) |
| B2M-cf1 gRNA | AGUGGGGGUGAAUUCAGUGU (SEQ ID NO: 5) |

TABLE 2
Primer/Probe Sequences

| Primer/Probe | Sequence |
|---|---|
| cssDNA Forward | TGGGCCATCGCCCTGATAGA |
| | (SEQ ID NO: 6) |
| cssDNA Reverse | AGAATAGCCCGAGATAGGGTTGAGT |
| | (SEQ ID NO: 7) |
| cssDNA probe | TCTTTAATAGTGGACTCTTGTTCCAAACT |
| | (SEQ ID NO: 8) |
| dsDNA Forward | GCTTAATCAGTGAGGCACCTATC |
| | (SEQ ID NO: 9) |
| dsDNA Reverse | GCCCTCCCGTATCGTAGTTAT |
| | (SEQ ID NO: 10) |
| dsDNA probe | CGTTCATCCATAGTTGCCTGACTCCC |
| | (SEQ ID NO: 11) |

### Enumerated Embodiments

Embodiment 1: A method of editing a DNA in cells, the method comprising delivering into the cells:
a circular single-stranded DNA (cssDNA) donor template comprising:
   a DNA insert;
   a 5′-homology arm; and
   a 3′-homology arm,
      wherein the 5′ homology arm and the 3′ homology arm are complementary to the DNA in a target region; and
a DNA editing construct, or a nucleic acid encoding the DNA editing construct,
   wherein the DNA editing construct has a site-specific DNA endonuclease activity, and
   wherein the DNA editing construct introduces a DNA break in the target region of the DNA,
wherein the DNA insert is inserted into the target region via homologous recombination (HR), or homology directed repair (HDR),
wherein the length of the DNA insert is about 2,000 bases or longer and editing efficiency is 20% or higher based on a total number of the cells, or the length of the DNA insert is less than about 2,000 bases and editing efficiency is 30% or higher, and
wherein the cells are at least one selected from the group consisting of induced pluripotent stem cells (iPSCs), hematopoietic stem cells (HSCs), B-cells, and T-cells.

Embodiment 2: The method of Embodiment 1, wherein at least one of the following applies:
(a) the cells are iPSCs, the length of the DNA insert is about 2,000 bases or longer, and the editing efficiency is 40% or higher based on a total number of the cells,
(b) the cells are hematopoietic stem cells (HSCs), the length of the DNA insert is about 2,000 bases or longer, and the editing efficiency is 40% or higher based on a total number of the cells,
(c) the cells are B-cells, the length of the DNA insert is about 2,000 bases or longer, and the editing efficiency is 20% or higher based on a total number of the cells,
(d) the cells are T-cells, the length of the DNA insert is about 2,000 bases or longer, and the editing efficiency is 40% or higher based on the total number of cells.

Embodiment 3: The method of any one of Embodiments 1-2, wherein each of the 5′-homology arm and the 3′-homology arm independently has a length ranging from about 25 nucleotides to about 5000 nucleotides.

Embodiment 4: The method of any one of Embodiments 1-3, wherein the DNA editing construct is or comprises at least one site-specific nuclease selected from the group consisting of a Cas nuclease, a zinc-finger nuclease, a meganuclease, and a transcription activator-like effector nuclease (TALEN).

Embodiment 5: The method of any one of Embodiments 1-4, wherein the DNA editing construct is or comprises a class I Cas protein or a class II Cas protein.

Embodiment 6: The method of any one of Embodiments 1-5, wherein the DNA editing construct is or comprises Cas9, Cas3, Cas10, Cas11, CasX, Cas12a, MAD7, or catalytic dead forms or nickase forms thereof.

Embodiment 7: The method of any one of Embodiments 1-6, wherein the DNA is a genomic DNA.

Embodiment 8: The method of any one of Embodiments 1-7, wherein the cells comprise two or more copies of the DNA, and wherein 20% or more of the edited cells are edited in two or more copies of the DNA.

Embodiment 9: The method of any one of Embodiments 1-8, wherein the construct and/or nucleic acid and the ssDNA are delivered into the cell by a viral vector, a lipid or non-lipid nanoparticle delivery, an exosome delivery, an electroporation delivery, a gene gun delivery, or an injection.

Embodiment 10: The method of any one of Embodiments 1-9, further comprising contacting the cells with a nonhomologous end joining (NHEJ) inhibitor or a DNA recombinase stimulator.

Embodiment 11: The method of Embodiment 10, wherein the NHEJ inhibitor is a DNA-dependent protein kinase (DNA-PK) inhibitor.

Embodiment 12: The method of Embodiment 11, wherein the DNA-PK comprises M-3814, Alt-R HDR enhancer, Alt-R HDR enhancer V2, generic DNA ligase inhibitor comprises SCR7, generic DNA recombinase stimulator comprises RS-1, or combinations thereof.

Embodiment 13: A kit for editing a DNA, comprising:
a circular single-stranded DNA (cssDNA) donor template comprising a DNA insert; a 5′-homology arm; and a 3′-homology arm, wherein the 5′ homology arm and the 3′ homology arm are complementary to the DNA in a target region;
a DNA editing construct or a nucleic acid encoding the DNA editing construct, wherein the DNA editing construct has a site-specific DNA endonuclease activity, and wherein the DNA editing construct introduces a DNA break in the target region of the DNA; and
a nonhomologous end joining (NHEJ) inhibitor or a DNA recombinase stimulator.

Embodiment 14: The kit of Embodiment 13, wherein each of the 5′-homology arm and the 3′-homology arm of the cssDNA donor template independently has a length ranging from about 25 nucleotides to about 5000 nucleotides.

Embodiment 15: The kit of any one of Embodiments 13-14, wherein the DNA editing construct is or comprises at least one site-specific nuclease selected from the group consisting of a Cas nuclease, a zinc-finger nuclease, a meganuclease, and a transcription activator-like effector nuclease (TALEN).

Embodiment 16: The kit of any one of Embodiments 13-15, wherein the DNA editing construct is or comprises a class I Cas protein or a class II Cas protein.

Embodiment 17: The kit of any one of Embodiments 13-16, wherein the DNA editing construct is or comprises Cas9, Cas3, Cas10, Cas11, CasX, Cas12a, MAD7, or catalytic dead forms or nickase forms thereof.

Embodiment 18: The kit of any one of Embodiments 13-17, wherein the DNA is a genomic DNA.

Embodiment 19: The kit of any one of Embodiments 13-18, wherein the NHEJ inhibitor is a DNA-dependent protein kinase (DNA-PK) inhibitor.

Embodiment 20: The kit of Embodiment 19, wherein the DNA-PK comprises M-3814, Alt-R HDR enhancer, Alt-R HDR enhancer V2, generic DNA ligase inhibitor comprises SCR7, generic DNA recombinase stimulator comprises RS-1.

Embodiment 21: The kit of any one of Embodiments 13-20, further comprising a component for delivering the DNA editing construct and/or the nucleic acid encoding the DNA editing construct, and the ssDNA, wherein the component comprises a microinjector, a lipid for forming lipid nanoparticles, a exosome, a gene gun, a metal nanoparticle, or a cuvette for electroporation.

Embodiment 22: A method of killing cancer cell, the method comprising contacting the cancer cell with an engineered T-cell comprising an engineered T-cell receptor (TCR) and/or a chimeric antigen receptor (CAR) targeting the cancer cell, wherein the engineered T-cell is prepared by delivering into a T-cell:
a circular single-stranded DNA (cssDNA) donor template comprising:
   a DNA insert;
   a 5′-homology arm; and
   a 3′-homology arm,
   wherein the 5′ homology arm and the 3′ homology arm are complementary to the DNA in a target region, and
a DNA editing construct or a nucleic acid encoding the DNA editing construct, wherein the DNA editing construct has a site-specific DNA endonuclease activity, and wherein the DNA editing construct introduces a DNA break in the target region of the DNA,
wherein the DNA insert is inserted into the target region via homologous recombination (HR), or homology directed repair (HDR).

Embodiment 23: The method of Embodiment 22, wherein
the method further comprises preparing the engineered T-cell, and
preparing the engineered T-cell comprises delivering into the T-cell the cssDNA donor template, and the DNA editing construct and/or the nucleic acid encoding the DNA editing construct.

Embodiment 24: The method of any one of Embodiments 22-23, wherein the insertion of the DNA insert produces a chimeric antigen receptor targeting at least one selected from the group consisting of B-cell maturation antigen (BCMA), carcinoembryonic antigen (CEA), CD2, CD4, CD5, CD7, CD19, CD22, CD30, claudin 18.2 (CLDN 18.2), epithelial cellular adhesion molecule (EpCAM), folate receptor 1 (FOLR1), human epidermal growth factor receptor 2 (HER2), mesothelin (MSLN), mucin 1 (MUC1), natural killer group 2D (NKG2D).

Embodiment 25: The method of any one of Embodiments 22-24, wherein the target region of the DNA is a B2M locus, a genomic safe harbor locus (such as an AAVS1 locus a CCR5 locus, a hRosa26 locus, a Rogi1 locus, a Rogi2 locus, a GSH1-6 locus, a PD1 locus, a TRAC locus, or combinations thereof.

Embodiment 26: The method of any one of Embodiments 22-25, wherein each of the 5′-homology arm and the 3′-homology arm independently has a length ranging from about 25 nucleotides to about 5000 nucleotides.

Embodiment 27: The method of any one of Embodiments 22-26, wherein the DNA editing construct is or comprises at least one site-specific nuclease selected from the group consisting of a Cas nuclease, a zinc-finger nuclease, a meganuclease, and a transcription activator-like effector nuclease (TALEN).

Embodiment 28: The method of any one of Embodiments 22-27, wherein the DNA editing construct is or comprises a class I Cas protein or a class II Cas protein.

Embodiment 29: The method of any one of Embodiments 22-28, wherein the DNA editing construct is or comprises Cas9, Cas3, Cas10, Cas11, CasX, Cas12a, MAD7, or catalytic dead forms or nickase forms thereof.

Embodiment 30: The method of any one of Embodiments 22-29, wherein the cancer cell is a leukemia cancer cell, such as an acute lymphoblastic leukemia cancer cell.

Embodiment 31: The method of any one of Embodiments 22-30, wherein the cancer cell is a cancer cell line, a primary cancer cell, or a cancer cell in the body of a subject.

Embodiment 32: The method of Embodiment 31, wherein the cancer cell is in the body of the subject, and wherein the subject is a mammal, such as a human.

Embodiment 33: A method of treating, ameliorating and/or preventing cancer in a subject in need thereof, the method comprising administering to the subject an effective amount of engineered T-cells comprising an engineered T-cell receptor or a chimeric antigen receptor targeting the cancer, wherein the CAR-T cell is prepared by delivering into a T-cell:
a circular single-stranded DNA (cssDNA) donor template comprising:
   a DNA insert;
   a 5′-homology arm; and
   a 3′-homology arm,
   wherein the 5′ homology arm and the 3′ homology arm are complementary to the DNA in a target region,
a DNA editing construct or a nucleic acid encoding the DNA editing construct, wherein the DNA editing construct has a site-specific DNA endonuclease activity, and wherein the DNA editing construct introduces a DNA break in the target region of the DNA,
wherein the DNA insert is inserted into the target region via homologous recombination (HR), or homology directed repair (HDR).

Embodiment 34: The method of Embodiment 33, wherein
the method further comprises preparing the engineered T-cells, and
preparing the engineered T-cells comprises delivering into the T-cell the cssDNA donor template, and the DNA editing construct and/or the nucleic acid encoding the DNA editing construct.

Embodiment 35: The method of any one of Embodiments 33-34, wherein the insertion of the DNA insert produces a chimeric antigen receptor targeting at least one selected from the group consisting of B-cell maturation antigen (BCMA), carcinoembryonic antigen (CEA), CD2, CD4, CD5, CD7, CD19, CD22, CD30, claudin 18.2 (CLDN 18.2), epithelial cellular adhesion molecule (EpCAM), folate receptor 1 (FOLR1), human epidermal growth factor receptor 2 (HER2), mesothelin (MSLN), mucin 1 (MUC1), natural killer group 2D (NKG2D).

Embodiment 36: The method of any one of Embodiments 33-35, wherein the target region of the DNA is a B2M locus, a genomic safe harbor locus (such as an AAVS1 locus, hRosa26, Rogi1, Rogi2, GSH1-6 and/or a CCR5 locus), a PD1 locus, a TRAC locus, or combinations thereof.

Embodiment 37: The method of any one of Embodiments 33-36, wherein each of the 5′-homology arm and the 3′-homology arm independently has a length ranging from about 25 nucleotides to about 5000 nucleotides.

Embodiment 38: The method of any one of Embodiments 33-37, wherein the DNA editing construct is or comprises at least one site-specific nuclease selected from the group consisting of a Cas nuclease, a zinc-finger nuclease, a meganuclease, and a transcription activator-like effector nuclease (TALEN).

Embodiment 39: The method of any one of Embodiments 33-38, wherein the DNA editing construct is or comprises a class I Cas protein or a class II Cas protein.

Embodiment 40: The method of any one of Embodiments 33-39, wherein the DNA editing construct is or comprises Cas9, Cas3, Cas10, Cas11, CasX, Cas12a, MAD7, or catalytic dead forms or nickase forms thereof.

Embodiment 41: The method of any one of Embodiments 33-40, wherein the cancer is a leukemia cancer, such as an acute lymphoblastic leukemia cancer or B-cell non-Hodgkin lymphoma.

Embodiment 42: The method of any one of Embodiments 33-41, wherein the subject is a mammal, such as a human.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. A method of editing a DNA in cells, the method comprising delivering into the cells:
a circular single-stranded DNA (cssDNA) donor template comprising:
a DNA insert;
a 5′-homology arm; and
a 3′-homology arm,
wherein the 5′ homology arm and the 3′ homology arm are complementary to the DNA in a target region; and
a DNA editing construct, or a nucleic acid encoding the DNA editing construct,
wherein the DNA editing construct has a site-specific DNA endonuclease activity, and
wherein the DNA editing construct introduces a DNA break in the target region of the DNA,
wherein the DNA insert is inserted into the target region via homologous recombination (HR), or homology directed repair (HDR),
wherein the length of the DNA insert is about 2,000 bases or longer and editing efficiency is 20% or higher based on a total number of the cells, or the length of the DNA insert is less than about 2,000 bases and editing efficiency is 30% or higher, and
wherein the cells are at least one selected from the group consisting of induced pluripotent stem cells (iPSCs), hematopoietic stem cells (HSCs), B-cells, and T-cells.

2. The method of claim 1, wherein at least one of the following applies:
(a) the cells are iPSCs, the length of the DNA insert is about 2,000 bases or longer, and the editing efficiency is 40% or higher based on a total number of the cells,
(b) the cells are hematopoietic stem cells (HSCs), the length of the DNA insert is about 2,000 bases or longer, and the editing efficiency is 40% or higher based on a total number of the cells,
(c) the cells are B-cells, the length of the DNA insert is about 2,000 bases or longer, and the editing efficiency is 20% or higher based on a total number of the cells,
(d) the cells are T-cells, the length of the DNA insert is about 2,000 bases or longer, and the editing efficiency is 40% or higher based on the total number of cells,
(e) each of the 5′-homology arm and the 3′-homology arm independently has a length ranging from about 25 nucleotides to about 5000 nucleotides,
(f) the DNA editing construct is or comprises at least one site-specific nuclease selected from the group consisting of a Cas nuclease, a zinc-finger nuclease, a meganuclease, and a transcription activator-like effector nuclease (TALEN),
(g) the DNA editing construct is or comprises a class I Cas protein or a class II Cas protein,
(h) the DNA editing construct is or comprises Cas9, Cas3, Cas10, Cas11, CasX, Cas12a, MAD7, or catalytic dead forms or nickase forms thereof,
(i) the DNA is a genomic DNA,
(j) the cells comprise two or more copies of the DNA, and 20% or more of the edited cells are edited in two or more copies of the DNA, or
(k) the construct or nucleic acid and the ssDNA are delivered into the cell by a viral vector, a lipid or non-lipid nanoparticle delivery, an exosome delivery, an electroporation delivery, a gene gun delivery, or an injection.

3-9. (canceled)

10. The method of claim 1, further comprising contacting the cells with a nonhomologous end joining (NHEJ) inhibitor or a DNA recombinase stimulator.

11. The method of claim 10, wherein the NHEJ inhibitor is a DNA-dependent protein kinase (DNA-PK) inhibitor.

12. The method of claim 11, wherein the DNA-PK comprises M-3814, Alt-R HDR enhancer, Alt-R HDR enhancer V2, generic DNA ligase inhibitor comprises SCR7, generic DNA recombinase stimulator comprises RS-1, or combinations thereof.

13. A kit for editing a DNA, comprising:
a circular single-stranded DNA (cssDNA) donor template comprising a DNA insert; a 5′-homology arm; and a 3′-homology arm, wherein the 5′ homology arm and the 3′ homology arm are complementary to the DNA in a target region;
a DNA editing construct or a nucleic acid encoding the DNA editing construct, wherein the DNA editing construct has a site-specific DNA endonuclease activity, and wherein the DNA editing construct introduces a DNA break in the target region of the DNA; and
a nonhomologous end joining (NHEJ) inhibitor or a DNA recombinase stimulator.

14. The kit of claim 13, wherein at least one of the following applies:
(a) each of the 5′-homology arm and the 3′-homology arm of the cssDNA donor template independently has a length ranging from about 25 nucleotides to about 5000 nucleotides,
(b) the DNA editing construct is or comprises at least one site-specific nuclease selected from the group consisting of a Cas nuclease, a zinc-finger nuclease, a meganuclease, and a transcription activator-like effector nuclease (TALEN),
(c) the DNA editing construct is or comprises a class I Cas protein or a class II Cas protein,
(d) the DNA editing construct is or comprises Cas9, Cas3, Cas10, Cas11, CasX, Cas12a, MAD7, or catalytic dead forms or nickase forms thereof,
(e) the DNA is a genomic DNA,
(f) the kit further comprises a component for delivering the DNA editing construct or the nucleic acid encoding the DNA editing construct, and the ssDNA, wherein the component comprises a microinjector, a lipid for forming lipid nanoparticles, a exosome, a gene gun, a metal nanoparticle, or a cuvette for electroporation.

15-18. (canceled)

19. The kit of claim 13, wherein the NHEJ inhibitor is a DNA-dependent protein kinase (DNA-PK) inhibitor.

20. The kit of claim 19, wherein the DNA-PK comprises M-3814, Alt-R HDR enhancer, Alt-R HDR enhancer V2, generic DNA ligase inhibitor comprises SCR7, generic DNA recombinase stimulator comprises RS-1.

21. (canceled)

22. A method of killing cancer cell, the method comprising contacting the cancer cell with an engineered T-cell comprising an engineered T-cell receptor (TCR) or a chimeric antigen receptor (CAR) targeting the cancer cell, wherein the engineered T-cell is prepared by delivering into a T-cell:
a circular single-stranded DNA (cssDNA) donor template comprising:
a DNA insert;
a 5′-homology arm; and
a 3′-homology arm,
wherein the 5′ homology arm and the 3′ homology arm are complementary to the DNA in a target region, and
a DNA editing construct or a nucleic acid encoding the DNA editing construct, wherein the DNA editing construct has a site-specific DNA endonuclease activity, and wherein the DNA editing construct introduces a DNA break in the target region of the DNA,
wherein the DNA insert is inserted into the target region via homologous recombination (HR), or homology directed repair (HDR).

23. The method of claim 22, wherein at least one of the following applies:
(a) the method further comprises preparing the engineered T-cell, wherein preparing the engineered T-cell comprises delivering into the T-cell the cssDNA donor template, and the DNA editing construct or the nucleic acid encoding the DNA editing construct,
(b) the insertion of the DNA insert produces a chimeric antigen receptor targeting at least one selected from the group consisting of B-cell maturation antigen (BCMA), carcinoembryonic antigen (CEA), CD2, CD4, CD5, CD7, CD19, CD22, CD30, claudin 18.2 (CLDN 18.2), epithelial cellular adhesion molecule (EpCAM), folate receptor 1 (FOLR1), human epidermal growth factor receptor 2 (HER2), mesothelin (MSLN), mucin 1 (MUC1), natural killer group 2D (NKG2D),
(c) the target region of the DNA is a B2M locus, a genomic safe harbor locus, a PD1 locus, a TRAC locus, or combinations thereof,
(d) each of the 5′-homology arm and the 3′-homology arm independently has a length ranging from about 25 nucleotides to about 5000 nucleotides,
(e) the DNA editing construct is or comprises at least one site-specific nuclease selected from the group consisting of a Cas nuclease, a zinc-finger nuclease, a meganuclease, and a transcription activator-like effector nuclease (TALEN),
(f) the DNA editing construct is or comprises a class I Cas protein or a class II Cas protein,
(g) the DNA editing construct is or comprises Cas9, Cas3, Cas10, Cas11, CasX, Cas12a, MAD7, or catalytic dead forms or nickase forms thereof,
(h) the cancer cell is a leukemia cancer cell, optionally an acute lymphoblastic leukemia cancer cell.

24-30. (canceled)

31. The method of claim 22, wherein the cancer cell is a cancer cell line, a primary cancer cell, or a cancer cell in the body of a subject.

32. The method of claim 31, wherein the cancer cell is in the body of the subject, and wherein the subject is a mammal, such as a human.

33. A method of treating, or ameliorating cancer in a subject in need thereof, the method comprising administering to the subject an effective amount of engineered T-cells comprising an engineered T-cell receptor or a chimeric antigen receptor targeting the cancer, wherein the CAR-T cell is prepared by delivering into a T-cell:
a circular single-stranded DNA (cssDNA) donor template comprising:
a DNA insert;
a 5′-homology arm; and
a 3′-homology arm,
wherein the 5′ homology arm and the 3′ homology arm are complementary to the DNA in a target region,
a DNA editing construct or a nucleic acid encoding the DNA editing construct, wherein the DNA editing construct has a site-specific DNA endonuclease activity, and wherein the DNA editing construct introduces a DNA break in the target region of the DNA,
wherein the DNA insert is inserted into the target region via homologous recombination (HR), or homology directed repair (HDR).

34. The method of claim 33, wherein at least one of the following applies:
(a) the method further comprises preparing the engineered T-cells, wherein preparing the engineered T-cells comprises delivering into the T-cell the cssDNA donor template, and the DNA editing construct or the nucleic acid encoding the DNA editing construct,
(b) the insertion of the DNA insert produces a chimeric antigen receptor targeting at least one selected from the group consisting of B-cell maturation antigen (BCMA), carcinoembryonic antigen (CEA), CD2, CD4, CD5, CD7, CD19, CD22, CD30, claudin 18.2 (CLDN 18.2), epithelial cellular adhesion molecule (EpCAM), folate receptor 1 (FOLR1), human epidermal growth factor receptor 2 (HER2), mesothelin (MSLN), mucin 1 (MUC1), natural killer group 2D (NKG2D),
(c) the target region of the DNA is a B2M locus, a genomic safe harbor locus, a PD1 locus, a TRAC locus, or combinations thereof,
(d) each of the 5′-homology arm and the 3′-homology arm independently has a length ranging from about 25 nucleotides to about 5000 nucleotides,
(e) the DNA editing construct is or comprises at least one site-specific nuclease selected from the group consisting of a Cas nuclease, a zinc-finger nuclease, a meganuclease, and a transcription activator-like effector nuclease (TALEN),
(f) the DNA editing construct is or comprises a class I Cas protein or a class II Cas protein,
(g) the DNA editing construct is or comprises Cas9, Cas3, Cas10, Cas11, CasX, Cas12a, MAD7, or catalytic dead forms or nickase forms thereof,
(h) the cancer is a leukemia cancer, optionally an acute lymphoblastic leukemia cancer or B-cell non-Hodgkin lymphoma,
(i) the subject is a mammal, or a human.

35-42. (canceled)


SEQUENCE LISTING (11 sequences)

SEQ ID NO: 1 - RNA, 20 nt, synthetic construct
  source 1..20: mol_type = other RNA
ggtagtcgtactcgtcgtcg

SEQ ID NO: 2 - RNA, 20 nt, synthetic construct
  source 1..20: mol_type = other RNA
ggccacggagcgagacatct

SEQ ID NO: 3 - RNA, 20 nt, synthetic construct
  source 1..20: mol_type = other RNA
agagtctctcagctggtaca

SEQ ID NO: 4 - RNA, 20 nt, synthetic construct
  source 1..20: mol_type = other RNA
ggggccactagggacaggat

SEQ ID NO: 5 - RNA, 20 nt, synthetic construct
  source 1..20: mol_type = other RNA
agtgggggtgaattcagtgt

SEQ ID NO: 6 - DNA, 20 bp, synthetic construct
  source 1..20: mol_type = other DNA
tgggccatcgccctgataga

SEQ ID NO: 7 - DNA, 25 bp, synthetic construct
  source 1..25: mol_type = other DNA
agaatagcccgagatagggttgagt

SEQ ID NO: 8 - DNA, 29 bp, synthetic construct
  source 1..29: mol_type = other DNA
tctttaatagtggactcttgttccaaact

SEQ ID NO: 9 - DNA, 23 bp, synthetic construct
  source 1..23: mol_type = other DNA
gcttaatcagtgaggcacctatc

SEQ ID NO: 10 - DNA, 21 bp, synthetic construct
  source 1..21: mol_type = other DNA
gccctcccgtatcgtagttat

SEQ ID NO: 11 - DNA, 26 bp, synthetic construct
  source 1..26: mol_type = other DNA
cgttcatccatagttgcctgactccc